(19) United States
(12) Patent Application Publication
(10) Pub. No.: US 2011/0269125 A1
(43) Pub. Date: Nov. 3, 2011

(54) METHODS AND COMPOSITIONS FOR PREDICTING DEVELOPMENT OF ATOPIC DISEASES

(76) Inventors: Mark Bix, Memphis, TN (US); Mariko Okamoto, Kanagawa (JP); Masato Kubo, Kanagawa (JP); Melanie Van Stry, Memphis, TN (US)

(21) Appl. No.: 12/990,669

(22) PCT Filed: May 7, 2009
(86) PCT No.: PCT/US09/02841

§ 371 Date: Jan. 10, 2011

Related U.S. Application Data
(60) Provisional application No. 61/051,554, filed on May 8, 2008.

(51) Int. Cl. C12Q 1/68

(52) U.S. Cl. 435/6.11

(57) ABSTRACT

The present invention relates to methods of testing for allergic diseases and methods for screening candidate compounds as therapeutic agents for allergic diseases. In particular the present invention provides genetic markers useful for the prediction of an individual's susceptibility to and/or the molecular diagnosis of atopic diseases such as bronchial asthma and allergic rhinitis.

This invention was made in part with government support under RO1 AI48636 awarded by the National Institutes of Health. As such the government has certain rights in the invention.

### FIELD OF THE INVENTION

The present invention relates to methods of testing for allergic diseases and methods for screening candidate compounds as therapeutic agents for allergic diseases. In particular the present invention provides genetic markers useful for the prediction of an individual's susceptibility to, and/or the molecular diagnosis of, atopic diseases such as bronchial asthma and allergic rhinitis.

### BACKGROUND OF THE INVENTION

The process for diagnosing an allergy has three principal components: (1) characterize the allergic reaction symptomatically, (2) correlate the reaction with antigen(s) to which the symptomatic subject has been exposed, and (3) demonstrate the presence of allergen-specific IgE antibodies. Symptoms that correlate with an exposure to an allergen and that manifest themselves beyond regions directly contacted by the allergen indicate the presence of atopic disease. Typically, the exposure elevates serum IgE. Atopic disease is also associated with a family history of allergic reactions to common environmental antigens. Approximately 30% of the population of the United States is estimated to be affected by allergic rhinitis, bronchial asthma or atopic dermatitis (Daffern and Schwartz, “Allergic Response,” in Dale and Federmen (eds.) ACP Medicine, WebMED Inc., 6(X):1-7, 2003). There is an increasing prevalence of allergic diseases in countries having a Western lifestyle. Many factors are likely to account for this increase, including a relative lack of microbial stimulation (Martinez, Respir Res, 2:129, 2001). This “hygiene hypothesis” takes into account that allergic diseases have both environmental and genetic influences.

To diagnose atopic disease, a patient's medical history is reviewed to establish a relationship between the time and site of allergen exposure and the development of allergic symptoms. Detection of allergen-specific IgE is done by in vivo and/or in vitro methods. Indirect determinations of IgE activity are made with skin tests. Skin testing can be done epicutaneously or intradermally. Both are rapid and sensitive but each has drawbacks. For instance, they rely on IgE inducing an elevation in histamine, so false negative results can occur if drugs having antihistamine properties (e.g., antihistamines and tricyclic antidepressants) are in use. In addition, the intradermal test, which can be regarded as highly invasive since it exposes the patient to a significant antigen load, poses an appreciable risk of a systemic reaction. Radioallergosorbent testing (RAST) and other in vitro tests directly measure IgE in serum and thus are not adversely affected by antihistamine use. RAST results generally correlate with allergic sensitivity, although they are more likely than skin testing to produce false positives.

In short, the commonly used in vivo and in vitro methods of diagnosing allergic disease have significant limitations. As such, tools for increasing the sensitivity or specificity of current diagnostic methods are needed in the art. Likewise, allergic testing methods that pose less risk to patients are desirable.

### SUMMARY OF THE INVENTION

The present invention relates to methods of testing for allergic diseases and methods for screening candidate compounds as therapeutic agents for allergic diseases. In particular, the present invention provides genetic markers useful for the prediction of an individual's susceptibility to or propensity for developing atopic disease. The markers also afford a new opportunity to diagnose, molecularly, atopic diseases such as bronchial asthma and allergic rhinitis.

In one embodiment, the invention provides a method of determining an individual's risk of developing an atopic disease comprising detecting, in a nucleic acid sample from said individual, the presence of at least one genetic variation, at least one of which is associated in linkage disequilibrium with at least a first MINA-associated single nucleotide polymorphism (“SNP”), wherein detection of the presence of the at least one linkage disequilibrium-associated genetic variation indicates that said individual has or is predisposed to the development of an atopic disease.

In one embodiment, the SNP is a minor allele selected from the group consisting of rs3886138, rs1532206 and rs2172257, wherein the presence of the minor allele indicates an increased risk for atopic disease relative to the presence of a major allele.

In one embodiment, the method comprises detecting said first MINA-associated SNP and further comprises detecting at least one other MINA-associated SNP wherein said first SNP and said at least one other SNP are in a haplotype, said haplotype comprising rs3886138G, rs1532206G and rs2172257T.

In one embodiment, the at least one genetic variation of the method consists of or comprises at least one copy of a human chromosome 3 comprising SNPs rs3886138G, rs1532206G and rs2172257T or SNPs in linkage disequilibrium therewith.

In one embodiment, the atopic disease to which the method applies is selected from the group consisting of bronchial asthma and allergic rhinitis.

In one embodiment, the nucleic acid sample comprises DNA. In another embodiment, the nucleic acid sample comprises RNA.

In one embodiment of the method, the detecting step is selected from the group consisting of size analysis (which may be preceded by a restriction enzyme digestion); sequencing; hybridization; 5′ nuclease digestion; single-stranded conformation polymorphism; allele specific hybridization; primer specific extension; and oligonucleotide ligation assay.

In one embodiment, the nucleic acid sample is amplified, optionally by means of a polymerase. In a particular embodiment, the amplification method detects at least one polymorphism.

In another embodiment, the at least one polymorphism is detected by sequencing.

In a preferred embodiment, the at least one polymorphism is detected by amplification of a target region containing the at least one polymorphism; and hybridization with at least one sequence-specific oligonucleotide that hybridizes under stringent conditions to the at least one polymorphism and detecting the hybridization.

In another aspect, the invention provides a method for selecting an appropriate therapeutic for an individual that has or is predisposed to developing an atopic disease. In one embodiment, the method comprises the steps of: detecting whether the subject contains a MINA-associated SNP; and selecting a therapeutic that compensates for the MINA-associated genetic variation. The detecting step may be conducted, without limitation, by allele specific oligonucleotide hybridization; size analysis; sequencing; hybridization; 5′ nuclease digestion; single-stranded conformation polymorphism; primer specific extension; and oligonucleotide ligation assay.

In one embodiment, the therapeutic is a modulator of MINA activity. In alternative embodiments, the modulator may be, without limitation, a protein, peptide, peptidomimetic, small molecule, nucleic acid or a nutraceutical.

In alternative embodiments, the detecting step of methods for selecting therapeutics is performed using a technique selected from the group consisting of allele specific oligonucleotide hybridization; size analysis; sequencing; hybridization; 5′ nuclease digestion; single-stranded conformation polymorphism; primer specific extension; and oligonucleotide ligation assay. In some embodiments, prior to or in conjunction with said detecting, the nucleic acid sample is subjected to an amplification step.

In yet another aspect, the invention provides a method of screening for predisposition to an atopic disease in a subject comprising detecting, in a nucleic acid sample from said individual, the presence of at least one genetic variation, at least one of which is associated in linkage disequilibrium with at least a first MINA-associated single nucleotide polymorphism (“SNP”), wherein said SNP is a minor allele selected from the group consisting of rs3886138, rs1532206 and rs2172257, wherein the presence of the minor allele indicates an increased risk for atopic disease relative to the presence of a major allele.

In one embodiment of the screening method, the sample is selected from the group consisting of blood, saliva, amniotic fluid, and tissue. In one embodiment, the nucleic acid is selected from the group consisting of mRNA, genomic DNA, and cDNA.

In still another aspect, the invention provides a kit for determining the existence of or a susceptibility to developing an atopic disease in a subject, said kit comprising a reagent for specifically detecting a specific MINA allele containing a SNP variation in linkage disequilibrium with at least a first MINA53-associated single nucleotide polymorphism (“SNP”).

In a preferred embodiment, the reagent comprises a first primer and a second primer that hybridize either 3′ or 5′ to the MINA gene, so that a polymorphism can be amplified. Preferably, the first primer and the second primer hybridize to a region in the range of between about 50 and about 1000 base pairs.

In one embodiment, the invention provides a kit for determining whether an individual's genome contains a specific MINA polymorphism and/or a specific haplotype in the proximity of the MINA gene. In some embodiments, the kit is useful in determining whether the subject is at risk for atopic disease. The diagnostic kits are produced in a variety of ways. In some embodiments, the kit contains at least one reagent for specifically detecting a specific MINA allele containing polymorphisms in the 3′ untranslated region and/or SNP polymorphisms in the MINA gene or its neighboring genes. In preferred embodiments, the reagents are primers for amplifying the region of DNA containing the polymorphism and/or SNP polymorphisms. In other preferred embodiments, the reagent is a probe that binds to the polymorphic region. In some embodiments, the kit contains instructions for determining whether the subject is at risk for atopic disease. In preferred embodiments, the instructions specify that risk for atopic disease is determined by detecting the presence or absence of a specific MINA allele in the subject, wherein subjects having a haplotype from the group consisting of a G at marker rs3886138; a G at marker rs1532206; and a T at marker rs2172257 are at risk. In some embodiments, the kits include ancillary reagents such as buffering agents, nucleic acid stabilizing reagents, protein stabilizing reagents, and signal producing systems (e.g., fluorescence generating systems). The test kit may be packaged in any suitable manner, typically with the elements in a single container or various containers as necessary along with a sheet of instructions for carrying out the test. In some embodiments, the kits also preferably include a positive control sample to ensure that the kit can in fact effect a measurement.

In one embodiment, the kit detects a polymorphism selected from the group consisting of rs3886138, rs1532206 and rs2172257. The kit may also comprise a detection means and/or an amplification means.

### DESCRIPTION OF THE FIGURES

FIG. 1 shows the narrowing of the Dice1.2 genetic interval. (A) C16D2/8D displays a low TH2 bias phenotype. Shown is relative IL4 expression (HPRT-normalized, arbitrary units) in activated CD4 T cells from individual BALB/c (BALB), B10.D2 (D2) and C16D2/8D mice. Reverse transcriptase real time PCR was used to quantitate RNA harvested from CD4-enriched splenic T cells stimulated for 16 hours with plate-bound antibodies to the T-cell receptor (TCR) and CD28. (B) Location of the mouse chromosome 16 congenic intervals in C16D2/8 and C16D2/8D. Shown for each strain are the chromosomal regions inherited from B10.D2 (grey), BALB/c (white) or flanking the congenic breakpoint (slashed). Genotyped locations are indicated with black dots. Genotyping markers and their locations are indicated on the right axis. The original and the new Dice1.2 genetic intervals are shown as long and short vertical bars.

FIG. 2 shows that Mina53 transcription is independent of and inversely correlated with IL4 expression. (A) Shown is IL4 and (B) Mina53 mRNA expression in CD4 T cells from low (B10.D2, C57BL/6, and C3H/HeN; grey lines and symbols) and high (BALB/c, DBA/1J, DBA/2J, and DBA/2N; black lines and symbols) TH2-biased strains stimulated with TCR cross-linking for the indicated times. Quantitative RT-PCR was used to determine the abundance of IL4 and Mina53 message in RNA extracted from CD4 T cells. Data are the average of three independent experiments and error bars indicate standard error of the mean. (C) Shown is Mina53 expression in CD4 T cells from BALB/c and B10.D2 stimulated with TCR cross-linking for 24 hr in the presence of the indicated amount of recombinant mouse IL4 (B10.D2) or anti-IL4 antibody (BALB/c). (D) Shown is Mina53 expression in CD4 T cells from B10.D2 stimulated for 24 hr in the presence of anti-IL12 antibody. Each bar graph represents the average of three independent experiments and error bars indicate SEM.

FIG. 3 shows that SNPs in the murine Mina53 locus define two haplotypes, correlating with TH2 bias. At the top is a schematic of the 5′ end of the murine Mina53 locus. Untranslated region (open) and coding (filled) exons are depicted as rectangles. Nucleotide positions relative to the translational start are indicated above, while SNP locations are indicated below the horizontal line. The table in the Figure shows the allele for each of 21 SNPs for five different strains, three with low (C57BL/6, B10.D2 and C3H/HeN) and two with high (BALB/c and DBA/2J) TH2 bias.

FIG. 4 shows that MINA53 binds to and represses transcription from the IL4 promoter. (A) Shows the transcriptional response to MINA53 of the IL4 and IL2 promoters. Luciferase or IL2 reporters were cotransfected with Mina53 or a control expression vector. Light emission was measured from unstimulated (open bars) and anti-TCR antibody (filled bars) stimulated transductants. Data are the average of three experiments and error bars indicate SEM (*p<0.05). (B) The nucleotide sequence of the proximal IL4 promoter along with the oligonucleotide probes (A, B, C, and D) used for EMSA is shown, along with polyacrylamide gels used to separate the EMSA products formed by combining extracts of cell nuclei from 24 hr TCR-stimulated B10.D2 CD4 T cells with the probes in the absence (left) or presence of an anti-MINA53 antibody (right). (C) Shown is a schematic of the IL4 promoter with regions interrogated by ChIP indicated by thin horizontal lines labeled with beginning and ending nucleotide coordinates (relative to the translation start site indicated by the arrow). Also shown is a graph depicting fold-enrichment for chromatin-bound MINA53 across the IL4 promoter and the CD3ε locus (negative control) in 24 hr TCR-stimulated CD4 T cells from BALB/c (open bars) and B10.D2 (filled bars) mice. Data shown are the mean of three independent experiments and error bars indicate SEM (*P, 0.05).

FIG. 5 shows that progenitor T cells from Mina53 transgenic mice exhibit impaired IL4 but not IL2 or IFNγ (interferon gamma) expression. Progenitor T helper cells isolated from three independent Mina53 transgenic lines (closed symbols representing numbers 6, 38 and 21) and their corresponding littermate controls (open symbols) were stimulated with antibodies to the TCR and CD28. RNA harvested at the indicated times post-stimulation was analyzed by reverse transcriptase-quantitative real time PCR. Data expressed as relative expression (β-actin normalized, arbitrary units) are from two independent experiments.

FIG. 6 shows that Mina53 is a differentially expressed gene in the Dice1.2 genetic interval as determined by comparative expression profiling of 30 of the 81 Dice1.2 candidate genes. mRNA from BALB/c and C16D2/8 CD4 T cells activated for 16 hrs by TCR cross-linking was analyzed by Affymetrix expression profiling chips.

FIG. 7 shows a quantitative ChIP plot depicting fold-enrichment of Mina53 across the murineIL4 locus, along with a schematic representing the IL4 locus. ChIP was performed with TCR-stimulated CD4 T cells from B10.D2 mice using a MINA53 specific antibody and PCR primers spanning the IL4 locus. In the schematic, horizontal arrows represent transcription units, tall black rectangles represent exons, arrowheads represent the location of DNAseI hypersensitive sites, and small rectangles numbered 1-32 represent the interrogated regions.

### DEFINITIONS

The practice of embodiments of the present invention, except where otherwise indicated, employs methods well known to practitioners in the arts of analytical biochemistry, microbiology, molecular biology and recombinant DNA techniques, and amply explained in the literature. To further facilitate an understanding of the present invention, a number of terms and phrases are defined below.

Abbreviations used herein are: kDa (kilodalton); rec. (recombinant); N (normal); M (molar); mM (millimolar); μM (micromolar); mol (moles); mmol (millimoles); μmol (micromoles); nmol (nanomoles); pmol (picomoles); g (grams); mg (milligrams); μg or ug (micrograms); ng (nanograms); l or L (liters); ml (milliliters); μl (microliters); cm (centimeters); mm (millimeters); μm (micrometers); nm (nanometers); ° C. (degrees Centigrade); hr (hours); mAb (monoclonal antibody); APC (antigen presenting cell); CTL (cytotoxic T lymphocyte); ChIP (chromatin immunoprecipitation); ELISA (enzyme-linked immunosorbent assay); EMSA (electrophoretic mobility shift assay); IL2 or IL-2 (interleukin-2); IL4 or IL-4 (interleukin-4); IP (immunoprecipitation); IFNγ (interferon-γ); MINA53 (Myc-Induced Nuclear Antigen Of 53 kilodaltons); PAGE (polyacrylamide gel electrophoresis); PCR (polymerase chain reaction); SEM (standard error of the mean); SNP (Single nucleotide polymorphisms); TCR (T cell receptor); TH (helper T); and UTR (untranslated region).

The term “subject” as used herein, refers to a mammal, preferably a human. It is intended that the term encompass healthy individuals as well as individuals predisposed to (or suspected of having) an allergic disease. Typically, the terms “subject,” “patient” and “individual” (when referring to a subject or a patient) are used interchangeably. In some preferred embodiments of the present invention, the term “subject” refers to specific subgroups of patients including, as non-limiting examples, subjects having positive RASTs for a dust mite allergen such as Derf (Dermatophagoides farinae) or Derp (Dermatophagoides pteronyssinus), but the term encompasses any living organism capable of mounting an immune response to an antigen. “RAST refers to the co-called “radioallergosorbent test,” which uses a radioactive tracer to measure the uptake of a patient's blood-borne antibodies by a suspected allergen.

As used herein, the term “diagnosis” refers to the determination of the nature of a case of disease. In some preferred embodiments of the present invention, methods for making a diagnosis are provided which permit atopic disease to be distinguished from other forms of allergic reactions. The term includes, but is not limited to any of the following: detection of an atopic disease that an individual may presently have, predisposition screening (i.e., determining the increased risk of an individual in developing an atopic disorder in the future, or determining whether an individual has a decreased risk of developing atopic disease in the future), determining a particular type or subclass of atopic diseases in an individual known to have atopia, confirming or reinforcing a previously made diagnosis of an atopia, pharmacogenomic evaluation of an individual to determine which therapeutic strategy that individual is most likely to positively respond to or to predict whether a patient is likely to respond to a particular treatment, predicting whether a patient is likely to experience toxic effects from a particular treatment or therapeutic compound, and evaluating the future prognosis of an individual having an atopic disorder. Such diagnostic uses are based on the SNPs individually or in a unique combination or SNP haplotypes of the present invention.

The term “therapy” (and variants thereof) as used herein refers to the treatment of a subject having a disease or a subject considered to be at risk of acquiring a disease either because the subject has a predisposition for the disease or because environmental conditions pose a risk. “Treatment” includes, without limitation, the administration of therapeutic or putatively therapeutic agents by any suitable route of administration, including but not limited to oral, parenteral, topical or by aerosol to the respiratory system. Effective treatment outcomes include, without limitation, reduction of symptoms associated with a disease; the prevention, delay of onset of, or acceleration of recovery from, a disease as determined by clinical symptoms, markers of the disease, or the subjective impression of an observer, including that of the treated subject. Exemplary markers may be apparent in amounts or turnover rates of enzymes, or products of such enzymes, that are linked to MINA expression, and factors that affect or are affected by such expression. Any such marker may be regarded herein as an indicator of “MINA activity, and any agent that affects any such marker is considered a “modulator” of MINA activity, whether the modulation is effected at the level of the gene (e.g., by “gene therapy”), or in connection with its expression or with the activity of its expression products. Where the term “MINA” or “MINA53” is used herein specifically to refer solely to a nucleic acid, the term will be modified accordingly (e.g., “MINA gene,” “MINA RNA”), or will be apparent from the context. Reference to the gene in the mouse will be made in lower case letters (i.e., “Mina”).

Modulators may include, without limitation, proteins, peptides, peptidomimetics, nucleic acids, small (i.e., non-polymeric) molecules, and nutritional agents (“nutraceuticals”), any of which may be delivered in any pharmaceutically acceptable formulation or, where suitable, in transplanted cells or tissues.

As used herein, the term “polymer” refers to a molecule comprising residues that occur repeatedly in the molecule. The repeating units need not be identical. Nucleic acid polymers, for example, typically comprise four different nucleotides, two of which comprise a purine and two of which comprise a pyrimidine.

The term “gene” refers to a polymer comprising nucleotides. In a functional gene, the nucleotides are arranged in a sequence that encodes for an amino acid sequence of a polypeptide or protein. A functional gene may or may not be expressed, however, depending upon a number of factors. Moreover, a gene need not be capable of functioning to be inherited. The nucleotide polymer is said to have a 5′ end and a 3′ end, a convenient reference that derives from the chemical structure of nucleotides. In general, nucleotide polymers that function as genes are deoxyribonucleic acids (DNA) or ribonucleic acids (RNA). The polypeptide can be encoded by a full-length coding sequence or by any portion of the coding sequence so long as the desired activity or functional properties (e.g., enzymatic activity, ability to bind to a binding partner or “ligand”, ability to participate in the transmission or “transduction” of a biochemical signal, ability to stimulate the immune system, etc.) of the “decoded” product are retained. The term also encompasses the coding region of a structural gene (encodes a protein that contributes primarily to the architecture of the cell) and the sequences located adjacent to the coding region on both the 5′ and 3′ ends for a distance of about 1 to 2 kb or more on either end. The exact range is determined by the nucleotide sequence transcribed into full-length messenger RNA (mRNA, sometimes referred to as “message,” is encoded in the DNA, but the code must be “transcribed” into RNA). Sequences located 5′ of the coding region (“upstream”) and present in the mRNA are referred to as 5′ non-translated sequences because they do not encode any elements of the protein that is ultimately expressed. Sequences located 3′ (“downstream”) of the coding region and present in the mRNA are referred to as 3′ non-translated sequences. These non-translated regions typically carry sites that contribute to the regulation of the actual expression of the gene by interacting with substances that promote or inhibit (repress, silence) transcription of the code from DNA to RNA. In RNA, the non-translated regions serve other regulatory functions such as a translation start site, where amino acid polymerization begins. A “gene” in its genomic form (so-called “genomic DNA”) contains coding regions (“exons”) interrupted or interspersed with non-coding sequences termed “introns,” “intervening regions” or “intervening sequences.” Nuclear RNA (hnRNA), the primary transcript, comprises both the coding sequences and the introns. Introns may also contain regulatory elements, such as enhancers, that interact with transcription factors. Introns are removed from hnRNA and the remaining (coding) segments are spliced together; introns therefore are absent in the messenger RNA (mRNA) transcript. The mRNA functions during translation to specify the sequence or order of amino acids in a nascent polypeptide.

As used herein, the term “nucleic acid” refers to any nucleic acid containing molecule, including but not limited to, DNA, cDNA and RNA. Unlike genomic DNA genes, a gene expressed as cDNA (“complementary DNA”) encompasses only those nucleotides that encode for mature mRNA. RNA differs from DNA in that its nucleotides comprise ribose instead of deoxyribose and one of the bases is uracil instead of thymine. In particular, the terms “MINA gene” and “MINA nucleic acid” refer to the full-length MINA nucleotide sequence (e.g., contained in human chromosome 3 from bp 99,173,984 to bp 99,143,347 (minus strand). The terms “MINA gene” and “MINA nucleic acid, as used herein, also encompass fragments of the MINA sequence, as well as other domains within the full-length MINA nucleotide sequence. Furthermore the term “MINA nucleotide sequence” encompasses DNA, cDNA, and RNA (e.g., GENBANK Accession No. AB083189) sequences. The name of the gene is a contraction of “Myc-Induced Nuclear Antigen,” so-called because a protein capable of raising antibodies (i.e., an “antigen”) is found in the nucleus of cells exposed to a transcription factor, called “Myc,” that mediates expression of a number of genes.

In addition to containing introns, genomic forms of a gene may also include sequences located on both the 5′ and 3′ end of the sequences that are not present on the RNA transcript. Referred to as “flanking” sequences or regions, they are located 5′ or 3′ to the non-translated sequences. The 5′ flanking region may, like other non-coding regions, contain regulatory sequences such as promoters and enhancers that control or influence the transcription of the gene. The 3′ flanking region may contain sequences that direct the termination of transcription, post-transcriptional cleavage (which ends transcription) and polyadenylation (which creates a protective “tail” for the mature mRNA).

As used herein, the term “portion of a chromosome” refers to any discrete section of a chromosome, however it may be delineated. Chromosomes are divided into sites or sections by cytogeneticists as follows: Chromosomes are paired (except for the Y chromosome, which “pairs” with an X chromosome). Each member contacts the other at a centromere from which a short arm termed the “p” arm, and a long arm termed the “q” arm, extend. Each arm is then divided into two regions termed region 1 and region 2. Region 1 is closest to the centromere. Each region is further divided into bands. The bands may be further divided into sub-bands. These sections and subsections of chromosomes are discernible by light microscopy. Resolving regions of chromosomes at the level of individual genes is accomplished indirectly, by determining sequences of nucleotides in a chromosome, and/or by analyzing the inheritance of particular genes to find, for example, genes (or other markers of specific locations on a chromosome) that stay together through meiosis (an event in inheritance that disrupts chromosomes internally and rearranges or “recombines” them) and are therefore close to one another on a chromosome (“linked”). Conversely, genes (or markers thereof) on a chromosome that tend to turn up independently in offspring are more weakly linked (i.e., farther apart). The resulting “linkage map” consigns genes to particular “genetic intervals” (such as Dice 1.2) on a chromosome (also referred to herein as a “locus”). A “congenic interval,” as that term is used herein, is a genetic interval found by interbreeding two inbred strains (the “recipient” and the “donor” strains) of mice (typically) and backcrossing certain of the offspring, selected by phenotype, until the strains become “congenic” (or “coisogenic”). Congenic strains have identical genomes except for one locus (“congenic interval”) responsible for the phenotype of interest (in this case, the Determinant of IL4 Commitment) and defined at each end by a so-called “congenic breakpoint.”

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in nucleotide sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product. Such mutants or variants may be referred to herein as “alleles,” a term coined when the physical nature of genes was unknown, used to explain the apparent inheritance of two “copies” of a gene whose interaction was not necessarily additive. The phenomenon is now understood to be rooted in subtle differences in the form of the inherited nucleic acid polymers (“polymorphism”). Thus, the term “allele,” as used herein, refers to one of at least two mutually exclusive forms of the same gene, occupying the same locus on homologous chromosomes, and governing the same biochemical and/or developmental process.

The term “genetic variation” relates to variations in the frequency with which one encounters a given gene (i.e., allele) in the genomes of different individuals or populations (defined by geography, race, ethnicity, etc.). The term is generally used herein to refer to a variation from wild-type or other predominant form of the gene. In contexts where the relative frequencies of two alleles are being compared, the less frequently encountered allele is referred to as the “minor allele” and the one more frequently encountered as the “major allele.”

The term “genotype” as used herein refers to the complete genetic code that is “written” in the chromosomes of every nucleated somatic cell of an individual but not necessarily expressed in any discernible way, as distinguished from an individual's or a cell's “phenotype,” which is an expression (at a molecular, cellular or even behavioral level) of the genotype. By way of pertinent but non-limiting example, an individual's T-helper cells all have the same genotype but that same individual has T-helper cells of at least two phenotypes. Cells of the T-helper 1 (“TH1”) phenotype, upon differentiating from multipotent progenitor T-helper cells, tend to mount an immune response to intracellular pathogens, whereas the T-helper 2 (“TH2”) phenotype responds to extracellular pathogens. Since both types circulate in any given individual, one speaks of the “bias” of the one population over the other. The individual is said to have either a TH2-bias phenotype or a TH1-bias phenotype.

In diploid organisms, chromosomes are paired. Each (“haploid”) member of a pair comprises a DNA polymer in the form of a double helix of nucleotides aligned in pairs across the helices. The base components of aligned nucleotides are referred to as “base pairs.” Each such double helix has its own genotype (the “haploid genotype” or “haplotype”). One expects these haplotypes, since they are in homologous chromosomes, to be highly similar but not necessarily identical. During ordinary cell division (mitosis), each haplotype is replicated, resulting in “sister chromatids” that are distributed intact to daughter cells. Such chromosome-length haplotypes do not necessarily survive meiosis, however, because segments (“blocks”) of paired haploid chromosomes tend to change places with one another (“recombine”) during meiosis in a process called “crossing over.” The genotype of the pre-meiotic arrangement of genes in the haploid chromosome survives within discrete blocks but the recombined post-meiotic chromosome displays, over its full-length, a different haplotype. A block may carry thousands of base pairs (and hundreds of genes), their sequence undisturbed by meiosis over many generations. Thus, the haplotype of the block is more stable genetically than the haplotype of the chromosome as a whole. The term “haplotype,” is often used to refer to the set of alleles (the nucleotide bases sited at a particular “address” or “co-ordinate” along a relevant DNA sequence) that comprise such a stable block of nucleotides. This meaning of “haplotype,” which is slightly different from the more general concept of a haplotype as an ordered array of alleles in a contiguous piece of DNA (including an entire chromosome), is generally used herein to describe embodiments of the present invention. In this context, a haplotype relates to a set of alleles of a group of closely linked genes, such as the HLA complex, which is usually inherited as a unit. The term also encompasses a set of markers of a group of closely linked markers. The term “marker” as used herein refers to an identifiable physical location on a chromosome (e.g., a specific site that is identified when it is “cut” or cleaved by a specific so-called “restriction” enzyme) whose inheritance can be monitored. Markers can be expressed regions of DNA (genes) or segments of DNA with no known coding function.

The invulnerability of haplotype blocks to variation does not extend to variations introduced into a block by mutation, 90% of which are expected to be “single nucleotide polymorphisms” or “SNPs.” As used herein, the term “polymorphism” refers to the regular and simultaneous occurrence in a single interbreeding population of two or more alleles of a gene, where the frequency of the rarer allele(s) is greater than can be explained by recurrent mutation alone (typically greater than 1%). A SNP in a haplotype block serves as a convenient marker of the inheritance of all the alleles in the block. Embodiments of the present invention take advantage of this convenience.

The genomes of all organisms undergo spontaneous mutation in the course of their continuing evolution, generating variant forms of progenitor genetic sequences. A variant form may confer an evolutionary advantage or disadvantage relative to a progenitor form or may be neutral. In some instances, a variant form confers an evolutionary advantage to the species and is eventually incorporated into the DNA of many or most members of the species and effectively becomes the progenitor form. Additionally, the effects of a variant form may be both beneficial and detrimental, depending on the circumstances. For example, a heterozygous sickle cell mutation confers resistance to malaria, but a homozygous sickle cell mutation is usually lethal. In many cases, both progenitor and variant forms survive and co-exist in a species population. The coexistence of multiple forms of a genetic sequence gives rise to genetic polymorphisms, including SNPs.

Approximately 90% of all polymorphisms in the human genome are SNPs. SNPs are single base positions in DNA at which different alleles, or alternative nucleotides, exist in a population. The SNP position (interchangeably referred to herein as SNP, SNP site, or SNP locus) is usually preceded by and followed by highly conserved sequences of the allele (e.g., sequences that vary in less than 1/100 or 1/1000 members of the populations). An individual may be homozygous or heterozygous for an allele at each SNP position. A SNP can, in some instances, be referred to as a coding SNP (“cSNP”) to denote that the nucleotide sequence containing the SNP is an amino acid coding sequence.

A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion or deletion variant referred to as an “indel.”

A synonymous codon change, or silent mutation/SNP (terms such as “SNP,” “polymorphism,” “mutation,” “mutant,” “variation,” and “variant” are used herein interchangeably, but it will be understood that a “SNP” is a special case thereof, wherein the mutation is due to the substitution of a single nucleotide), due to the degeneracy of the genetic code, does not result in a change of amino acid in the peptide that the SNP-containing gene encodes. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. While SNPs can be bi-, tri-, or tetra-allelic, the vast majority of the SNPs are bi-allelic, and are thus often referred to as “bi-allelic markers,” or “di-allelic markers.”

As used herein, references to “SNPs” and SNP genotypes include individual SNPs and/or haplotypes, meaning groups of SNPs that are generally inherited together. Haplotypes can have stronger correlations with diseases or other phenotypic effects compared with individual SNPs, and therefore may provide increased diagnostic accuracy in some cases.

“Causative SNPs” produce alterations in gene expression or in the expression, structure, and/or function of a gene product, and therefore are most predictive of a possible clinical phenotype. One such class includes SNPs falling within a coding region of a gene, or “cSNPs.” These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic disease. Examples of genes in which a SNP within a coding sequence causes a genetic disease include sickle cell anemia and cystic fibrosis.

Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions. Some SNPs that are not causative SNPs nevertheless are in close association with, and therefore segregate with, a disease-causing sequence. In this situation, the presence of a SNP correlates with the presence of, or predisposition to, or an increased risk in developing the disease. These SNPs, although not causative, are nonetheless also useful for diagnostics, disease predisposition screening, and other uses.

An association study of a SNP and a specific disorder involves determining the presence or frequency of the SNP allele in biological samples from individuals with the disorder of interest, such as an atopia, and comparing the information to that of controls (i.e., individuals who do not have the disorder; controls may be also referred to as “healthy” or “normal” individuals) who are preferably of similar age and race. The appropriate selection of patients and controls is important to the success of SNP association studies. Therefore, a pool of individuals with well-characterized phenotypes is extremely desirable.

A SNP may be screened in diseased tissue samples or any biological sample obtained from a diseased individual, and compared to control samples, and selected for its increased (or decreased) occurrence in a specific pathological condition, such as pathologies related to asthma or rhinitis. Once a statistically significant association is established between one or more SNP(s) and a pathological condition (or other phenotype) of interest, then the region around the SNP can optionally be thoroughly screened to identify the causative genetic locus/sequence(s) (e.g., causative SNP/mutation, gene, regulatory, region, etc.) that influences the pathological condition or phenotype. Association studies can be conducted within the general population and are not limited to studies performed on related individuals in affected families (linkage studies).

Clinical trials have shown that patient response to treatment with pharmaceuticals is often heterogeneous. There is a continuing need to improve pharmaceutical agent design and therapy. In that regard, SNPs can be used to identify patients most suited to therapy with particular pharmaceutical agents (this is often termed “pharmacogenomics” or “pharmacogenetics”).

Similarly, SNPs can be used to exclude patients from certain treatment due to the patient's increased likelihood of developing toxic side effects or their likelihood of not responding to the treatment. Pharmacogenomics can also be used in pharmaceutical research to assist the drug development and selection process.

Cells of the immune system are (properly) quiescent unless “stimulated” or “activated” by a challenge (e.g., an antigen). As used herein, activation may also be mediated experimentally using, for example, an antibody to the T-cell receptor.

As used herein, the term “DNaseI hypersensitive site” refers to a genomic region occurring in chromatin (a complex of DNA and proteins) as it exists natively within the nucleus, the region being characterized by its marked sensitivity in a “DNaseI hypersensitivity assay” to digestion by the enzyme DnaseI. Such hypersensitive regions correlate with elements in the DNA that interact with regulatory signals responsible for controlling the expression of genes.

As used herein the term “ChIP” refers to “Chromatin ImmunoPrecipitation,” a well-known technique for determining whether a specific biomolecule is associated with a particular genomic region occurring in chromatin as it exists natively within the nucleus.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “complementary” or “complementarity” are used in reference to polynucleotides (i.e., a sequence of nucleotides, each of which comprises a nitrogenous base bonded to a sugar moiety) related by the base-pairing rules. For example, for the sequence “A-G-T,” is complementary to the sequence “T-C-A.” Complementarity may be “partial,” in which only some of the nucleic acid's bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “Southern blot,” refers to the analysis of DNA on agarose or acrylamide gels used to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58, 1989).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52, 1989).

Gel electrophoresis of DNA and RNA to detect protein-DNA or protein-RNA interactions is routinely employed in Electrophoretic Mobility Shift Assays (“EMSA”) wherein the DNA or RNA fragment and the protein of interest (or a control) are electrically driven through a gel. If the protein is capable of binding to the fragment, a less mobile complex of nucleic acid probe bound to protein will result and will be shifted up on the gel. In some embodiments an antibody to the protein is included to perform a so-called “supershift” assay.

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization, 1985). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” or “target regions” in the sense that they are sought to be sorted out from other nucleic acid. Amplification techniques have been designed primarily for this sorting out.

As used herein, the term “sample template” refers to nucleic acid originating from a sample that is analyzed for the presence of “target.” In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). Primers are designed to be “sequence-specific.” That is, they are selected on the basis of their propensity for hybridizing to a target sequence. The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “sense primer” refers to an oligonucleotide capable of hybridizing to the noncoding strand of gene. The term “antisense primer” refers to an oligonucleotide capable of hybridizing to the coding strand of a gene.

As used herein, the term “fluorescent tag” refers to a molecule having the ability to emit light of a certain wavelength when activated by light of another wavelength. “Fluorescent tags” suitable for use with the present invention include but are not limited to fluorescein, rhodamine, Texas red, 6-FAM, TET, HEX, Cy5, Cy3, and Oregon Green.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “polymerase chain reaction” (“PCR”) refers to the method of Mullis U.S. Pat. Nos. 4,683,195 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; inclusion of dye-labeled nucleotides in the reaction and their incorporation into the PCR product; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” as used herein, refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

An alternative PCR method, “real-time PCR” employs fluorescent dyes or other markers that permit quantitative measurement of the reaction with each reaction cycle. “Reverse transcriptase PCR,” begins with an RNA template from which copies of cDNA are made under catalysis by the enzyme reverse transcriptase. The cDNA so produced is then employed in traditional PCR (or real-time PCR) to effect further amplification. Reverse transcriptase PCR performed in combination with real-time PCR is a powerful means for making quantitative measurements of mRNA.

As used herein, the terms “ligase chain reaction” and “ligase amplification reaction” refer to methods for detecting small quantities of a target DNA, with utility similar to PCR. Ligase chain reaction relies on DNA ligase to join adjacent synthetic oligonucleotides after they have bound the target DNA. Their small size means that they are destabilized by single base mismatches and so form a sensitive test for the presence of mutations in the target sequence.

The terms “single-strand conformation polymorphism” and “SSCP,” as used herein, refer to the ability of single strands of nucleic acid to take on characteristic conformations under non-denaturing conditions, which in turn can influence the electrophoretic mobility of the single-stranded nucleic acids. Changes in the sequence of a given fragment (i.e., mutations) will also change the conformation, consequently altering the mobility and allowing this to be used as an assay for sequence variations (Orita et al., Genomics 5:874-879, 1989).

As used herein, the terms “conformation-sensitive gel electrophoresis” or “CSGE” refer to methods for detecting mutations involving distinguishing DNA heteroduplexes from homoduplexes via mildly denaturing gel electrophoresis. CSGE protocols are well known in the art (Ganguly et al., Proc Natl Acad Sci USA 90:10325-10329, 1993).

The term “sequencing” refers to methods used to determine the order of nucleotide bases in a DNA or RNA molecule or fragment. The term includes for example, dideoxy sequencing and Maxam-Gilbert sequencing.

The terms “5′ nuclease digestion” and “restriction enzyme digestion” refer to well-known methods for cutting strands of nucleic acids into segments for further analysis. The size of the segments, and their sequences, for example, provide data useful in identifying the sequence of nucleotides in the larger nucleic acid. A number of well-known physical methods, including but not limited to gel electrophoresis, are employed in “size analysis.”

“Single-stranded conformation polymorphism” refers to a well-known electrophoretic method that has sufficient resolution to distinguish between SNPs.

“Allele specific hybridization” is a technique that relies on the selection of nucleic acid probes that anneal specifically to a particular allele.

“Primer specific extension” is a widely used technique that takes advantage of the “proofreading” function of 3′->5′ exonuclease to distinguish among alleles. The exonuclease must “see” the correct allele to permit a primer to “grow” or extend on a DNA template.

The “oligonucleotide ligation assay” uses a pair of oligonucleotide probes (oligomers) that hybridize to adjacent segments of DNA including the variable base. The oligomer on the 5′ or left-hand side of the pair is an allele-specific oligonucleotide (ASO) to one allele of the target. The last base at the 3′ end of this ASO is positioned at the site of the target DNA's polymorphism; the ASO also has a biotin molecule at its 5′ end that functions as a chemical hook. The oligomer on the 3′ or right-hand side of the pair is the common oligomer (sequence is the same for the two different alleles.) The common oligomer is positioned at an invariable site next to the target DNA's polymorphism and is fluorescently labeled at its 3′ end.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness (e.g., an allergic reaction), sickness, or disorder of bodily function. Test compounds comprise both known and potential therapeutic compounds. A test compound can be determined to be therapeutic by screening using the screening methods of the present invention.

The term “change” as used herein refers to a difference or a result of a modification or alteration. In preferred embodiments, the term “change” refers to a measurable difference between states (e.g., MINA mRNA or MINA protein expression in a cell in the presence and absence of a test compound). In some embodiments, the change is at least 10%, preferably at least 25%, more preferably at least 50%, and most preferably at least 90% more or less than that of a control condition. In preferred embodiments, measurements of change take into account variations between compared individuals. For example, mRNA expression is normalized internally to either β-actin or hypoxanthine-guanine phosphoribosyl transferase (“Hprt”), both of which are found in abundance in virtually all cells.

“Linkage disequilibrium” (LD) refers herein to the co-inheritance of alleles (e.g., alternative nucleotides) at two or more different SNP (or other allele marker) sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium.” In contrast, LD refers to any non-random genetic association between allele(s) at two or more different SNP sites. Although LD is driven, in part, by linkage (two or more loci whose residence on a single chromosome survives meiosis), the concept encompasses associations among sites on different chromosomes. In any event, some alleles tend to remain unseparated for multiple generations with the consequence that a particular allele at one site will show a non-random association with a particular allele at a different site. This linkage disequilibrium means that genotyping one of the sites (for example, a SNP at the site) will give almost the same information as genotyping the other site that is in LD with the first site.

As used herein the term “comparative expression profiling” refers to a method in which an array of genes in each of two or more subjects are compared to determine which, if any, of the arrayed genes are expressed differently in the two subjects. In preferred embodiments, a very large array of small samples of the genes of interest (a “microarray”) is used as a standard.

As used herein, the term “transgenic” refers to a cell or organism into which a gene is introduced in a functional form, usually through the germline but, for some purposes, directly into somatic cells.

As used herein, the term “sample” is meant to include a specimen obtained from subject. The term “sample” encompasses fluids, solids, and tissues. In preferred embodiments, the term “sample” refers to blood or biopsy material obtained from a living body for the purpose of examination via any appropriate technique (e.g., needle, sponge, scalpel, swab, etc.).

As used herein, the terms “bronchial asthma” and “BA” refer to a disease of the lungs in which an obstructive ventilation disturbance of the respiratory passages evokes a feeling of shortness of breath. The cause is a sharply elevated resistance to airflow in the airways. The elevation may be secondary to an allergic response to a specific allergen.

As used herein, the terms “allergic rhinitis” and “AR” refer to an allergic reaction to an allergen that causes inflammation of the nasal membranes, sneezing, nasal congestion, nasal itching, and rhinorrhea. The eyes, ears, sinuses, and throat can also be involved. It is an extremely common condition, affecting approximately 20% of the population. While allergic rhinitis is not a life-threatening condition, complications can occur and the condition can significantly impair quality of life, which leads to a number of indirect costs. The total direct and indirect cost of allergic rhinitis was recently estimated to be $5.3 billion per year.

As used herein, the term “risk of developing atopic disease” refers to a subject's relative risk (e.g., the percent chance or a relative score) of developing an atopic disease during their lifetime.

The term “reagent(s) capable of specifically detecting a polymorphism in a MINA allele” refers to reagents used to detect the polymorphism in question from a MINA gene, cDNA, or RNA. Examples of suitable reagents include but are not limited to, nucleic acid probes capable of specifically hybridizing to MINA mRNA or cDNA.

As used herein, the term “instructions for determining whether a subject is predisposed to atopic disease” refers to instructions for using the reagents contained in the kit for the detection and characterization of an allele of a MINA gene in a sample from a subject. In some embodiments, the instructions further comprise the statement of intended use required by the U.S. Food and Drug Administration (FDA) in labeling in vitro diagnostic products: The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination. Additional information is available at the Internet web page of the U.S. FDA.

### GENERAL DESCRIPTION OF THE INVENTION

Bronchial asthma and allergic rhinitis are globally important diseases associated with elevated interleukin-4 (IL4) production from dysregulated T helper 2 (TH2) cell responses to innocuous environmental antigens. While applicants will not be bound by any theory or explanation of how embodiments of the present invention work, it is generally understood that a low-level of IL4, when rapidly elevated in the course of progenitor T helper cell activation, acts by positive feedback to instruct differentiation of the progenitor cells into mature high-level IL4-producing TH2 cells. In mice, Dice1.2 has been identified as an IL4 regulatory locus controlling the propensity of progenitor T helper cells to differentiate into TH2 cells. As determined during development of the present invention, MINA53, a member of the Jumonji protein family having a molecular weight of 53 kilodaltons, and mapping to Dice1.2, binds to and inhibits transcription from the IL4 promoter in progenitor T helper cells, thereby inhibiting TH2 cell differentiation and accounting for strain specific differences in TH2 bias. Further, as described herein, single nucleotide polymorphisms (SNPs) in the human MINA locus (orthologous to murine Mina53) are associated with bronchial asthma (BA) and allergic rhinitis (AR), two classic TH2 diseases. Together, these findings reveal a connection between TH2 regulation and the development of atopic diseases.

### DETAILED DESCRIPTION OF THE INVENTION

The present invention relates to methods of testing for allergic diseases and methods for screening candidate compounds as therapeutic agents for allergic diseases. In particular, the present invention provides genetic markers useful for the prediction of an individual's susceptibility to and/or the molecular diagnosis of atopic diseases such as bronchial asthma and allergic rhinitis.

### I. T Helper 2 (TH2) Cell Responses

As previously mentioned, explanations of how embodiments of the present invention work are not intended to be limiting. When provided herein, the intent is only to assist the reader to better understand the various embodiments. In this connection, progenitor T helper cells may be viewed as multipotent sentinels of the immune system, poised to take instructive signals from cells adapted to “present” antigens to them (“antigen-presenting dendritic cells”). The progenitor cells respond to such instructions (i.e, become “activated”) by differentiating into at least 4 distinct effector T helper cell lineages. These include the T helper 1 (TH1) and T helper 2 (TH2) lineages specialized, respectively, for the control of intra- and extra-cellular pathogens. Dysregulated development of TH2 cells can lead to pathological outcomes such as allergic rhinitis and bronchial asthma, in which even the most innocuous environmental antigens elicit inappropriate and harmful immune responses. Development of TH2 cells depends upon a positive feedback signaling loop in which low but functionally critical levels of IL4 produced by progenitor (or “naïve”) T helper cells activate a pathway (the so-called IL4R/STAT6/GATA3 pathway) within those very progenitor cells. The pathway's activation induces the cells to differentiate into TH2 cells. The IL4 functions analogously to an endocrine hormone that stimulates its own secretion (ergo “autocrine”).

The genetic pathway that regulates the initial expression of IL4 from progenitor T helper cells has not been elucidated fully. Several recent reports have revealed that, in addition to signals from the T cell receptor (TCR) and from CD28 (a receptor which, when activated by antigen-presenting cells, potentiates the T-cell receptor pathway to “co-stimulate” the T-cells), IL4 expression from progenitor T helper cells requires activation of the Notch pathway. Notch is a nearly ubiquitous transmembrane protein that, upon interacting with signaling molecules outside the cell, loses its intracellular portion. The released portion reaches the nucleus and binds to a hypersensitive region in the chromatin (HS V) that then enhances transcription of IL4 (unpublished observations of applicants). Indeed, progenitor T helper cells that lack HS V are actually incapable of producing IL4 and hence cannot develop into TH2 cells, suggesting that Notch-activated HS V, more than merely enhancing IL4 transcription, may be required to activate the IL4 promoter (unpublished observations).

The foregoing lends credence to the notion that the pathology of bronchial asthma and allergic rhinitis is a consequence of defective regulation of the physiology of TH2-cells, a defect manifested either as an excessive number of TH2 cells or as an excessive potency of the cells, or both. It is not known if TH2-cell dysregulation in fact accounts for atopic allergies and, if so, how such dysregulation has its impact. The objects of embodiments of the invention, however, are to provide tests to diagnose allergic diseases and methods for screening candidate compounds as therapeutic agents for allergic diseases. It will be understood that how or why an inventive device or a process works need not be known to give effect to such device or process.

### II. Inhibition of IL4 Expression by MINA, a Jumonji Family Nucleoprotein

A cell's ability to respond positively to feedback from IL4 is innate in the cell's genome. Whether or not the cell will so respond, however, depends on genetic regulation, wherein DICE appears to play an inhibitory role. The DICE 1.2 locus was originally identified by interval specific congenic mapping as one of two independent loci underlying DICE, a quantitative trait locus mapping to murine chromosome 16 that explained ˜30% of the TH2 bias variance in a [(BALB/c×C57BL/6)×C57BL/6] backcross cohort (Bix et al., J Exp Med, 188:2289, 1998). Multiple independent lines of evidence determined during development of the present invention indicate that Mina53 is DICE1.2. First, Mina53 maps to the minimal Dice 1.2 genetic interval. Second, MINA53 levels correlate inversely with TH2 bias. Third, both Dice 1.2 and Mina53 influence the level of HA expressed by progenitor T helper cells upon in vitro activation. MINA53 levels differ in progenitor T helper cells taken from high vs. low TH2 biased strains and the addition of exogenous IL4 or removal of endogenous IL4 from cells of either strain doesn't alter this difference, indicating that MINA53 operates in an IL4 production pathway upstream of the IL4 signaling-dependent TH2 commitment pathway, consistent with a causative role in influencing TH2 bias. Fourth, non-coding SNPS at the Mina53 locus define two distinct haplotypes that correlate with MINA53 and IL4 expression level in progenitor T helper cells. Fifth, IL4 but not IL2 expression is blunted in Mina53 transgenic progenitor T helper cells. Sixth, in transient reporter assays (which measure promoter activity) MINA53 can inhibit expression from an IL4 but not an IL2 promoter. Finally, MINA53 binds in vivo to the IL4 promoter at levels that correlate with its differential expression in high vs. low TH2 biased strains, indicating that IL4 expression is a bona fide target of MINA53.

Recent observations by the applicants lend further support to this assertion and may also provide insight into how antigen-presenting cells drive differentiation of progenitor T cells. The applicants have discovered that Mina53 levels (RNA transcript and protein) are high in C57BL/6 and low in BALB/c bone marrow derived dendritic cells (BMDCs), correlating inversely with their respective capacity to instruct nTh to differentiate into Th2 cells. To test whether this inverse correlation arises from a functional role of Mina53 as a regulator of BMDC Th2-inducing capacity, the applicants compared C57BL/6-background littermate control WT and Mina53 knockout (KO) mice. The results show that, compared to WT control, Mina53 KO BMDCs have a 5-10 fold higher capacity to drive nTh cells to become Th2 cells. Based on these results applicants conclude that Mina53 acts as a repressor of the Th2-inducing capacity of BMDCs and that natural variation in the level of Mina53 expression in BMDCs is a significant factor determining the propensity to mount Th2-biased immune responses. Thus, Mina53 acts via independent mechanisms delivered in two distinct cell types (nTh cells and BMDCs) to achieve repression of Th2-biased immune responses.

Twenty-one SNPs from the Mina53 promoter and intron 1 define two haplotypes that correlate with MINA53 and IL4 expression levels and TH2 bias. One or several of these SNPs in the DNA double helix in which they reside are contemplated to perturb key regulatory elements in that strand (so-called “cis regulatory elements”), altering transcription factor binding and thereby modulating MINA53 expression levels. Transcriptional induction of MINA53 was reported to require binding of the transcription factor Myc to two sites on the region of Mina53 that binds Mina53's promoter. The sites, called E-boxes, are within exon 1b of the Mina53 locus (Tanaka et al., Immunity, 24:689, 2006). However, as these E-box sites are conserved among the mouse strains examined, it is unlikely that differential sensitivity to MYC-dependent transcriptional induction is responsible for differential MINA53 levels in high and low TH2 biased strains. In any case, as noted above, the mechanism by which an inventive device or a process works need not be known to give effect to such device or process.

MINA53 belongs to the Jumonji (Jmj) protein family whose hallmark JmjC domain has been found recently to possess histone demethylase activity in a number of Jmj family members (Klose et al., Nat Rev Genet, 7:715, 2006). Consistent with a role in transcriptional regulation, MINA53 protein localizes predominantly to the nucleus and is especially concentrated in the nucleolus (Tsuneoka et al., J Biol Chem, 277:35450, 2002). It is not known whether the JmjC domain of MINA53 possesses histone demethylase activity or whether such an activity is responsible for its IL4 inhibitory effect. Preliminary immunofluorescence analysis indicates that MINA53 overexpression is insufficient to diminish total cellular levels of H3K4me3 and H3K4me2 and a JmjC deletion mutant of MINA53 is still capable of inhibiting IL4 expression in a reporter assay. As the IL4 promoter region spanning −94 to −65 (where MINA53 binds) includes a binding site for a transcription factor called “nuclear factor of activated T cells” (NFAT) (Szabo et al., Mol Cell Biol, 13:4793, 1993; and Chuvpilo et al., Nucl Acids Res, 21:5694, 1993), another model to explain the inhibitory effect of MINA53 on IL4 transcriptional activity is binding site competition with NFAT. Nonetheless knowledge of the mechanism(s) involved is not necessary in order to make and use the present invention.

The orthologous human gene, MINA, as described in Example 2, is shown to be associated with bronchial asthma, hyper-serum IgE and allergic rhinitis (e.g., hallmarks of dysregulated TH2 immunity). Thus MINA is contemplated to bind to the IL4 promoter in progenitor T helper cells thereby negatively regulating its activity. It follows that the genetically determined level to which MINA can be induced controls the extent of TH2 differentiation. Thus, individuals whose levels are low are predisposed to atopic disease. MINA and its regulatory pathway are contemplated to be ideal targets for the development of therapeutics capable of influencing TH2 development and ameliorating atopic disease.

### III. Detection of MINA Alleles

A. Single Nucleotide Polymorphisms (SNPs) In MINA Alleles

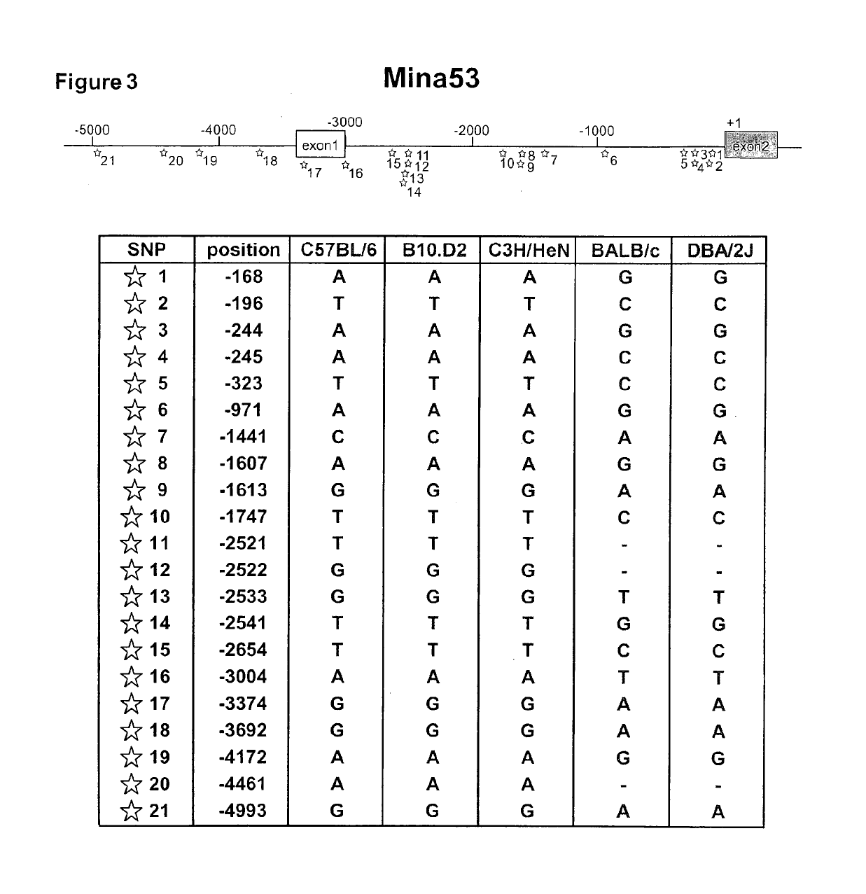

In some embodiments, the present invention includes alleles of MINA that increase a subject's tendency to develop atopic diseases such as bronchial asthma and allergic rhinitis. A non-limiting example is rs3886138, associated with susceptibility to childhood atopic bronchial asthma and to allergic rhinitis. Another is rs832082, associated with both childhood and adult bronchial asthma. Another is rs1532206, associated with childhood atopic bronchial asthma, adult bronchial asthma and adult atopic bronchial asthma. However, the present invention is not limited to these polymorphisms. In fact, any MINA polymorphism and any polymorphism in linkage with a MINA polymorphism that is associated with atopic diseases are within the scope of the present invention. Indeed, one or more of at least 21 SNPs from the MINA promoter and intron 1 (which together define two haplotypes that correlate with MINA and IL4 expression levels and TH2 bias) are useful in various embodiments. (See, FIG. 3).

B. Detection of MINA Alleles

Accordingly, the present invention provides methods for determining whether a patient has an increased susceptibility to one or more precipitating factors in atopic disease by determining whether the individual has a particular MINA allele. In other embodiments, the present invention provides methods for providing a prognosis of increased risk for atopic disease based on the presence or absence of one or more polymorphisms in the MINA gene. In preferred embodiments, the polymorphism causes or contributes to the etiology of atopic disease.

A number of methods are available for analysis of polymorphisms. Assays for detection of polymorphisms or mutations fall into several categories, including, but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention.

### 1. Direct Sequencing Assays

In some embodiments of the present invention, polymorphisms are detected using a direct sequencing technique. In these assays, DNA samples are first isolated from a subject using any suitable method. In some embodiments, the region of interest is cloned into a suitable vector and amplified by growth in a host cell (e.g., a bacteria). In other embodiments, DNA in the region of interest is amplified using PCR.

Following amplification, DNA in the region of interest (e.g., the region containing the polymorphism of interest) is sequenced using any suitable method, including but not limited to manual sequencing using radioactive marker nucleotides, or automated sequencing. The results of the sequencing are displayed using any suitable method. The sequence is examined and the presence or absence of a given polymorphism is determined.

### 2. PCR Assay

In some embodiments of the present invention, polymorphisms are detected using a PCR-based assay. In some embodiments, the PCR assay comprises the use of oligonucleotide primers to amplify a MINA fragment containing the polymorphism of interest. In other embodiments, the PCR assay comprises the use of oligonucleotide primers that hybridize only to a specific polymorphism in an allele of MINA (e.g., to the region of polymorphism). Both sets of primers are used to amplify a sample of DNA. If only the primers complementary to a particular polymorphism result in a PCR product, then the patient has that polymorphism. In other embodiments, the PCR reaction contains nucleotides labeled with dye or radioactive material.

### 3. Fragment Length Polymorphism Assays

In some embodiments of the present invention, polymorphisms are detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing a polymorphism will have a different banding pattern than wild type.

a. RFLP Assay

In some embodiments of the present invention, polymorphisms are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from wild-type and mutant controls.

b. CFLP Assay

In other embodiments, polymorphisms are detected using a CLEAVASE fragment length polymorphism assay (CFLP; Third Wave Technologies, Madison, Wis.; See e.g., U.S. Pat. No. 5,888,780). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given SNP or mutation. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

### 4. Hybridization Assays

In preferred embodiments of the present invention, polymorphisms are detected by hybridization assay. In a hybridization assay, the presence or absence of a given polymorphism or mutation is determined based on the ability of the DNA from the sample to hybridize to a complementary DNA molecule (e.g., a oligonucleotide probe). A variety of hybridization assays using a variety of technologies for hybridization and detection are available. A description of a selection of assays is provided below.

a. Direct Detection of Hybridization

In some embodiments, hybridization of a probe to the sequence of interest (e.g., polymorphism) is detected directly by visualizing a bound probe (e.g., a Northern or Southern assay; See e.g., Ausabel et al. (eds.), Current Protocols in Molecular Biology, John Wiley & Sons, NY, 1991). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., agarose gel electrophoresis) and transferred to a membrane. A labeled (e.g., by incorporating a radionucleotide) probe or probes specific for the mutation being detected is allowed to contact the membrane under a condition of low, medium, or high stringency conditions. Unbound probe is removed and the presence of binding is detected by visualizing the labeled probe.

b. Detection of Hybridization Using “DNA Chip” Assays

In some embodiments of the present invention, polymorphisms are detected using a DNA chip hybridization assay. In this assay, a series of oligonucleotide probes are affixed to a solid support. The oligonucleotide probes are designed to be unique to a given polymorphism. The DNA sample of interest is contacted with the DNA “chip” and hybridization is detected.

In some embodiments, the DNA chip assay is a GeneChip (Affymetrix, Santa Clara, Calif.; See e.g., U.S. Pat. No. 6,045,996) assay. The GeneChip technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, the identity of the target nucleic acid applied to the probe array can be determined by complementarity.

In other embodiments, a DNA microchip containing electronically captured probes (Nanogen, San Diego, Calif.) is utilized (See e.g., U.S. Pat. No. 6,068,818). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (See e.g., U.S. Pat. No. 6,001,311). Protogene's technology is based on the fact that fluids can be segregated on a flat surface by differences in surface tension that have been imparted by chemical coatings. Once so segregated, oligonucleotide probes are synthesized directly on the chip by ink jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removed by spinning.

DNA probes unique for the polymorphism of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or mutation. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization

In some embodiments of the present invention, genomic profiles are generated using a assay that detects hybridization by enzymatic cleavage of specific structures (INVADER assay, Third Wave Technologies; See e.g., U.S. Pat. No. 6,001,567). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the de-quenched fluorescein labeled product may be detected using a standard fluorescence plate reader.

The INVADER assay detects specific mutations and SNPs in unamplified genomic DNA. The isolated DNA sample is contacted with the first probe specific either for a SNP/mutation or wild type sequence and allowed to hybridize. Then a secondary probe, specific to the first probe, and containing the fluorescein label, is hybridized and the enzyme is added. Binding is detected using a fluorescent plate reader and comparing the signal of the test sample to known positive and negative controls.

In some embodiments, hybridization of a bound probe is detected using a TaqMan assay (PE Biosystems, Foster City, Calif.; See e.g., U.S. Pat. No. 5,962,233). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; See e.g., U.S. Pat. No. 5,952,174). In this assay, SNPs are identified using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin).

### 5. Mass Spectroscopy Assay

In some embodiments, a MassARRAY system (Sequenom, San Diego, Calif.) is used to detect polymorphisms (See e.g., U.S. Pat. No. 6,043,031). DNA is isolated from blood samples using standard procedures. Next, specific DNA regions containing the polymorphism of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

### 6. Methods for Selecting and Identifying SNPs and Identifying Haplotypes

SNPs are identified from both the dbSNP and the Applied Biosystems databases. Genotyping is carried out using TaqMan SNP Genotyping Assays (Applied Biosystems) according to the manufacturers' protocol for the snips listed in Table 1:

TABLE 1
SNPS, and Exemplary Primers and Probes for
Their Isolation and Identification

| | | |
|---|---|---|
| Name | SEQ ID | SEQ ID |

Human genotyping

| | | | | |
|---|---|---|---|---|
| | Forward primer | | Reverse primer | |
| rs3886138 | CAGCATCATTGTGACTGGAGGTA | 01 | TCCAGCCTGGGCATCAG | 02 |
| rs9877544 | TGTCAGACCAAGTTATGATTTGAATTCAGA | 03 | CAAGGACTAGCTCAGCGAGAAG | 04 |
| rs1532206 | CCCTCCGGGTTGAGACTCA | 05 | CCTGGCGGCCTCACT | 06 |
| rs832082 | CCAGCCTGTTATTTCTTCTCTGTCT | 07 | TGGGAATTGCCTACACAAAATGGATAT | 08 |
| rs2172257 | TGTTGTTGATGGCCTAATGCA | 09 | CCCATCATGTGTGTCTCTCTACTATTC | 10 |
| | FAM probe | | VIC probe | |
| rs3886138 | AAAGTGCAACCCTGTCCT | 11 | AAAGTGCAACCCTATCCT | 12 |
| rs9877544 | TTTTATTGACGGTGTCCAT | 13 | CATTTTATTGACAGTGTCCAT | 14 |
| rs1532206 | TTTGTGACTTAATGTTATATC | 15 | CTTTGTGACTTAATATTATATC | 16 |
| rs832082 | AACACGGGAAGAAG | 17 | TGATAACACAGGAAGAAG | 18 |
| rs2172257 | CAAAGGATGAAACTCAAGAA | 19 | AAAGGATGAAGCTCAAGAA | 20 |

Haplotype blocks can be identified using Haploview version 3.2 (Barrett et al., Bioinformatics, 21:263, 2005), a software program that employs a two-marker expectation-maximization (EM) algorithm to estimate maximum likelihood values for deriving an estimate of linkage disequilibrium (D′). Haplotype blocks can be identified based on established criteria (Gabriel et al., Science, 296:2225, 2002). Haplotypes can be ascertained for each individual using PHASE version 2.1 (Stephens et al., Am J Hum Genet, 68:978 2001; and Stephens and Donnelly, Am J Hum Genet, 73:1162, 2003), which utilizes a Bayesian statistical method for reconstructing haplotypes from population data. This software can handle SNP and microsatellite data, and can compensate for missing data. Haplotype phase probabilities for individuals can also be calculated using the I function in the “haplo.stat” package in R, available from the Mayo Clinic College of Medicine, Schaid Lab website.

### 7. Kits for Analyzing Risk of Atopic Disease

The present invention also provides kits for determining whether an individual's genome contains a specific MINA polymorphism and/or a specific haplotype in the proximity of the MINA gene. In some embodiments, the kits are useful in determining whether the subject is at risk for atopic disease. The diagnostic kits are produced in a variety of ways. In some embodiments, the kits contain at least one reagent for specifically detecting a specific MINA allele containing polymorphisms in the 3′ untranslated region and/or SNP polymorphisms in the MINA gene or its neighboring genes. In preferred embodiments, the reagents are primers for amplifying the region of DNA containing the polymorphism and/or SNP polymorphisms. In other preferred embodiments, the reagent is a probe that binds to the polymorphic region. In some embodiments, the kit contains instructions for determining whether the subject is at risk for atopic disease. In preferred embodiments, the instructions specify that risk for atopic disease is determined by detecting the presence or absence of a specific MINA allele in the subject, wherein subjects having a haplotype from the group consisting of a G at marker rs3886138; a G at marker rs1532206; and a T at marker rs2172257 are at risk. In some embodiments, the kits include ancillary reagents such as buffering agents, nucleic acid stabilizing reagents, protein stabilizing reagents, and signal producing systems (e.g., fluorescence generating systems). The test kit may be packaged in any suitable manner, typically with the elements in a single container or various containers as necessary along with a sheet of instructions for carrying out the test. In some embodiments, the kits also preferably include a positive control sample.

### 8. Bioinformatics

In some embodiments, the present invention provides methods of determining an individual's risk for developing an atopic disease based on the presence of one or more specific alleles of MINA as described in section 6. In other embodiments, the information on the presence or absence of one or more specific alleles of MINA is combined with data on the presence or absence of other polymorphisms in the human genome for determining an individual's risk of developing an atopic disease. In some embodiments, the analysis of polymorphism data is automated. For example, in some embodiments, the present invention provides a bioinformatics research system comprising a plurality of computers running a multi-platform object oriented programming language (See e.g., U.S. Pat. No. 6,125,383). In some embodiments, one of the computers stores genetics data (e.g., the risk of developing an atopic disease associated with a given polymorphism). In some embodiments, one of the computers stores application programs (e.g., for analyzing transmission disequilibrium data or determining genotype relative risks and population attributable risks). Results are then delivered to the user (e.g., via one of the computers or via the internet).

IV. Uses of SNPs and Haplotypes Associated with Atopic Disease.

In one aspect, the invention provides methods or screening assays for identifying modulators, i.e., candidate or test compounds or agents (e.g., peptides, peptidomimetics, small molecules, ribozymes, or MINA antisense molecules) which bind to MINA proteins, have a stimulatory or inhibitory effect on expression of MINA or on MINA activity, or have a stimulatory or inhibitory effect on the expression or activity of a MINA target molecule. Compounds identified using the assays described herein may be useful for treating atopic diseases.

Haplotypes are particularly useful in that, for example, fewer SNPs need to be genotyped to determine if a particular genomic region harbors a locus that influences a particular phenotype, such as in linkage disequilibrium-based SNP association analysis. For diagnostic applications, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., bronchial asthma, allergic rhinitis) that is influenced by the causative SNP(s). Thus, polymorphic markers that are in LD with causative polymorphisms are useful as diagnostic markers, and are particularly useful when the actual causative polymorphism(s) is/are unknown.

Practitioners skilled in the treatment or diagnosis of allergic disorders will understand that the information derived from using the methods of the invention is extremely valuable as it can be used, for example, to initiate preventive treatments or to allow an individual carrying one or more significant SNPs or SNP haplotypes to foresee warning signs such as minor clinical symptoms, or to have regularly scheduled physical exams to monitor for appearance of a condition in order to identify and begin treatment of the condition at an early stage (e.g., children).

The present invention also provides methods for assessing the potential response of a subject harboring particular SNP alleles or haplotypes to a particular therapeutic agent or pharmaceutical compound, or to a class of such compounds.

Pharmacogenomics deals with the roles which clinically significant hereditary variations (e.g., SNPs) play in the response to drugs due to altered drug disposition and/or abnormal action in affected persons. The clinical outcomes of these variations can result in severe toxicity of therapeutic drugs in certain individuals or therapeutic failure of drugs in certain individuals as a result of individual variation in metabolism. Thus, the SNP genotype of an individual can determine the way a therapeutic compound acts on the body or the way the body metabolizes the compound. For example, SNPs in drug metabolizing enzymes can affect the activity of these enzymes, which in turn can affect both the intensity and duration of drug action, as well as drug metabolism and clearance.

The SNPs of the present invention also can be used to identify novel therapeutic targets for atopic diseases. For example, genes containing the disorder associated variants or their products, as well as genes or their products that are directly or indirectly regulated by or interacting with these variant genes or their products, can be targeted for the development of therapeutics that, for example, treat the disorder or prevent or delay disease onset. The therapeutics may be composed of, for example, small molecules, proteins, protein fragments or peptides, antibodies, nucleic acids, or their derivatives or mimetics which modulate the functions or levels of the target genes or gene products.

The SNP-containing nucleic acid molecules disclosed herein, and their complementary nucleic acid molecules, may be used as antisense constructs to control gene expression in cells, tissues, and organisms. The SNPs of the present invention are particularly useful for designing antisense reagents that are specific for particular nucleic acid variants. Based on the SNP information disclosed herein, antisense oligonucleotides can be produced that specifically target mRNA molecules that contain one or more particular SNP nucleotides. In this manner, expression of mRNA molecules that contain one or more undesired polymorphisms (e.g., SNP nucleotides that lead to a defective protein such as an amino acid substitution in a catalytic domain) can be inhibited or completely blocked. Thus, antisense oligonucleotides can be used to specifically bind a particular polymorphic form (e.g., a SNP allele that encodes a defective protein), thereby inhibiting translation of this form, but which do not bind an alternative polymorphic form (e.g., an alternative SNP nucleotide that encodes a protein having normal function).

The SNPs of the present invention are also useful for designing RNA interference reagents that specifically target nucleic acid molecules having particular SNP variants. RNA interference (RNAi), also referred to as gene silencing, is based on using double-stranded RNA (dsRNA) molecules to turn genes off. As with antisense reagents, RNAi reagents may be directly useful as therapeutic agents (e.g., for turning off defective, disease-causing genes), and are also useful for characterizing and validating gene function (e.g., in gene knock-out or knock-down experiments).

The invention further provides a method for identifying a compound or agent that can be used to treat an atopic disorder. The SNPs disclosed herein are useful as targets for the identification and/or development of therapeutic agents. A method for identifying a therapeutic agent or compound typically includes assaying the ability of the agent or compound to modulate the activity and/or expression of a SNP-containing nucleic acid or the encoded product and thus identifying an agent or a compound that can be used to treat a disorder characterized by undesired activity or expression of the SNP-containing nucleic acid or the encoded product. The assays can be performed in cell-based and cell-free systems. Cell-based assays can include cells naturally expressing the nucleic acid molecules of interest or recombinant cells genetically engineered to express certain nucleic acid molecules.

Variant gene expression in a patient with an atopic disorder can include, for example, either expression of a SNP-containing nucleic acid sequence (for instance, a gene that contains a SNP can be transcribed into an mRNA transcript molecule containing the SNP, which can in turn be translated into a variant protein) or altered expression of a normal/wild-type nucleic acid sequence due to one or more SNPs (for instance, a regulatory/control region can contain a SNP that affects the level or pattern of expression of a normal transcript).

Evidence that the MINA gene controls TH2 bias includes the identification of the Mina53 as a gene in the murine chromosome 16 Dice 1.2 genetic interval that is also an interleukin-4 (IL4 or IL-4) regulatory locus. The several lines of supporting evidence adduced below are in no way intended to limit the scope of any invention described herein. On the contrary, the disclosures below may teach or suggest additional embodiments and it is to be understood that any such embodiments are within the scope of the invention.

The evidence was obtained as follows:

1. Establishment of congenic mouse strains and phenotypic screening for TH2 cell bias. Generation of congenic strain C16D2/8 was described previously (Baguet et al., J Exp Med, 200:1605-1612, 2004). First BALB.D2c16 mice were generated from a founding (BALB/c×B10.D2) F1 backcrossed to BALB/c and containing a B10.D2 derived congenic interval spanning the majority of chromosome 16. Nine congenic lines (C16D2/1-9) were generated by backcrossing BALB.D2c16 to BALB/c. After screening these congenic lines for the TH2 cell bias trait, C16D2/8 was selected for further analysis. Backcrossing C16D2/8 to BALB/c generated c16D2/8-a and C16D2/8-b congenic lines. Splenic CD4 T cells, isolated by MACS CD4 T cell Isolation kit from Miltenyi Biotec (Auburn, Calif.), were stimulated with plate-bound anti-TCR (1 μg/ml) and anti-CD28 (37N.51.1, 10 μg/ml) antibodies for 16 hours prior to harvesting RNA with TRI REAGENT from Sigma (St. Louis, Mo.) and determining Il4 and Hprt expression by reverse transcriptase real time PCR as described (Baguet et al., Proc Natl Acad Sci USA, 101:11410-11415, 2004). In subsequent experiments, splenic CD4 T cells were isolated by iMag CD4 T cell Isolation kit from Sigma, and stimulated with plate-bound anti-TCR (1 μg/ml) and soluble anti-CD28 (PV-1, 10 μg/ml) antibodies for 24 hours prior to harvesting RNA with TRI REAGENT from Sigma, and expression analysis by reverse transcriptase real time PCR as described (Seki et al., J Immunol, 172:6158-6166, 2004).

2. Generation of Mina53 Transgenic Mice. To establish Mina53 transgenic mice, a B10.D2 Mina53 cDNA was cloned downstream of the Lck proximal promoter and Eμ enhancer in the p1026X expression vector (Attar et al., Mol Cell Biol, 18:477-487, 1998). PCR of genomic tail DNA identified ten independent BDF1XB6 transgenic founders. Transgene copy number was determined by Southern blot analysis. Three founders harboring over 10 copies of the transgene were selected and backcrossed for three generations to BALB/c prior to further analysis. Protein expression in splenic CD4 T cells was confirmed by immunoblot analysis with anti-MINA53 antibody.

3. Reagents and antibodies. Anti-TCRβ antibody was purified from hybridoma H57.597. Anti-CD28 was purified, respectively, from hybridomas PV-1 and 37N51.1, kind gifts respectively from Ryo Abe (Science University of Tokyo) and James Allison (Memorial Sloan Kettering Cancer). Anti-IL4 was used as 10% culture supernatant from hybridoma 11B11, a kind gift from William Paul (NIH). Anti-IL12 was used as 10% culture supernatant from hybridoma C17.8, a kind gift from Georgio Trinchieri (NCI). Anti-MINA53 antibody (clone M532 described by Teye et al., Am J Pathol, 164:205-216, 2004; and Tsuneoka et al., Clin Cancer Res, 10:7347-7356, 2004) was purchased from Zymed (South San Francisco, Calif.).

4. Preparation of RNA and expression profiling. CD4 T cells were isolated from BALB/c or congenic strains, and cells were stimulated with plate bound anti-TCR mAb (H57-597, 1 μg/ml) and soluble anti-CD28 mAb (37N.51.1, 10 μg/ml). After 24 hrs, total RNA was extracted with TRI REAGENT from Sigma and passed through RNeasy mini-columns from QIAGEN (Valencia, Calif.). cRNA were synthesized from the isolated RNA according to Affymetrix protocols. The biotinylated cRNAs were hybridized to Affymetrix Inc. (Santa Clara, Calif.) GeneChips (mouse Genome 430 version 2). The microarray data were globally normalized using the GeneSpring software package from Agilent Technologies (Santa Clara, Calif.).

5. Quantitative RT-PCR Total RNA was extracted from cells and cDNA was synthesized using SuperScript III Reverse Transcriptase from Invitrogen Corp. (Carlsbad, Calif.). Real-time quantitative reverse transcription PCR analysis was performed on MX4000 from Stratagene (La Jolla, Calif.) and 7500 real-time PCR systems from Applied Biosystems (Foster City, Calif.). The PCR primers used are shown in Table 1-1. mRNA expression was normalized internally to either β-actin or Hprt.

6. Transient reporter assay. Mouse (strain B10.D2) Mina53 cDNA was inserted into pCMV10 from Sigma-Aldrich to generate the pCMV FLAG-Mina53 expression construct. T cell hybridoma, 68-41 cells were transfected with the IL4 or IL2 promoter luciferase reporter plasmid (pIL4 Lu or pIL2 Lu) in the presence of pCMV or pCMV FLAG-Mina53, and a pancreatic alkaline phosphatase (PAP) expression plasmid (pSVPAP) to control for transfection efficiency. Forty hrs after transfection, cells were stimulated for 12 hrs with plate-bound anti-TCR mAb and cell lysates were prepared by extraction with lysis buffer from Promega Corp. (Madison, Wis.). Luciferase activity measured with a Microplate luminometer (Promega) was normalized to PAP activity.

7. Electrophoretic mobility shift assay (EMSA). Nuclear proteins were extracted with CelLytic Nuclear Extraction Kit from Sigma. The nuclear proteins (10 μg) were incubated with 1×10⁴ cpm of end-labeled oligonucleotide probe for 30 min at room temperature. The samples were loaded on 4% polyacrylamide gel and run in low ionic strength buffer (1×TAE). The supershift assay was carried out by incubation with rabbit anti-MINA53 antibody.

8. Chromatin immunoprecipitation (ChIP) assay. The ChIP assay was performed as previously described (Koyanagi et al., J Biol Chem, 280:31470-31477, 2005). Briefly, DNA recovered from an aliquot of sheared chromatin was used as the input sample. The remaining chromatin was pre-cleared with protein A and protein G agarose from Amersham Biosciences (Piscataway, N.J.) and then incubated with anti-MINA Ab overnight at 4° C. Input DNA and DNA recovered after immunoprecipitation (IP) were quantified with a PicoGreen fluorescence assay kit from Molecular Probes (Eugene, Oreg.). Equivalent masses of IP and input DNA were compared by real time PCR. Data are presented as the ratio of IP to input CT values.

TABLE 1-1
Primers and probes

| | | |
|---|---|---|
| Name | SEQ ID | SEQ ID |

Human genotyping

| | | | | |
|---|---|---|---|---|
| | Forward primer | | Reverse primer | |
| rs3886138 | CAGCATCATTGTGACTGGAGGTA | 01 | TCCAGCCTGGGCATCAG | 02 |
| rs9877544 | TGTCAGACCAAGTTATGATTTGAATTCAGA | 03 | CAAGGACTAGCTCAGCGAGAAG | 04 |
| rs1532206 | CCCTCCGGGTTGAGACTCA | 05 | CCTGGCGGCCTCACT | 06 |
| rs832082 | CCAGCCTGTTATTTCTTCTCTGTCT | 07 | TGGGAATTGCCTACACAAAATGGATAT | 08 |
| rs2172257 | TGTTGTTGATGGCCTAATGCA | 09 | CCCATCATGTGTGTCTCTCTACTATTC | 10 |
| | FAM probe | | VIC probe | |
| rs3886138 | AAAGTGCAACCCTGTCCT | 11 | AAAGTGCAACCCTATCCT | 12 |
| rs9877544 | TTTTATTGACGGTGTCCAT | 13 | CATTTTATTGACAGTGTCCAT | 14 |
| rs1532206 | TTTGTGACTTAATGTTATATC | 15 | CTTTGTGACTTAATATTATATC | 16 |
| rs832082 | AACACGGGAAGAAG | 17 | TGATAACACAGGAAGAAG | 18 |
| rs2172257 | CAAAGGATGAAACTCAAGAA | 19 | AAAGGATGAAGCTCAAGAA | 20 |

Mouse genotyping

| | | | | |
|---|---|---|---|---|
| | Forward primer | | Reverse primer | |
| SNP59.49 | ACTCATCACTAGGTGAGCGACACT | 21 | AGGCGTGTATGTGTCAGCCTTTCT | 22 |
| SNP59.54 | TAACAGCAAGCCACTCTTCGTCCT | 23 | TTGCCACCATGTCTTTGCTCTGTG | 24 |
| MB04 | AATATCTTTGGGCTTCCTACTGC | 25 | TTCAATTGCTTTCATTTGGATCT | 26 |

ChIP Expression

| | | | | |
|---|---|---|---|---|
| 1 | TCCTGTGAATCCTTCATGTCAAGC | 27 | AAGTGTCATCATCGTCCTGGAGC | 28 |
| 2 | CAATCTTGTTCCCCCAGTAG | 29 | AAGATGGAAATCATGCCAGT | 30 |
| 3 | GTCCTCTTATCGACCCCATC | 31 | AAAGGCTTGGGGAAACAC | 32 |
| 4 | GTCTATATCCCTCCCACTCGT | 33 | GGGCAGGTGAGTATCAGTCTA | 34 |
| 5 | TCAAGCCTGATGACCTATGC | 35 | AGGGTCTCCAGAAGATTAGCC | 36 |
| 6 | GAATGCCAGTGCTCTTACAGT | 37 | CATGCCCAGTTGAGATTGT | 38 |
| 7 | GCAAAAGAAACTCTCAGCAAGGCC | 39 | CCGTCCCTCTCGCCTTCAGT | 40 |
| 8 | CGAGGCCTCATTATCTTCAT | 41 | GGAGATGACGAGGTTTGCTA | 42 |
| 9 | TGCATGTACTTTGGTAGGGTC | 43 | AGAGTGGCACCTGAGACTTAG | 44 |
| 10 | TCCAGCCTCTGCCTTATC | 45 | CTGGAGCAAAGAATAGACCAT | 46 |
| 11 | CAAAGAGAGACCCGGTCTCC | 47 | GGAGGTTCTGGGCTAGGTTG | 48 |
| 12 | AGGTAAGACCCCAGAGTCAGCTTTC | 49 | TCAGCGTAGGGTTGCCACTG | 50 |
| 13 | CACTTGAGAGAGATCATCGG | 51 | CCACCTCTCTAGCAACTCAG | 52 |
| 14 | GCCTTGCTTGATACGGTATCT | 53 | GCCTGTAGGGACCATACGA | 54 |
| 15 | AGGCCTCTTTCTGGTCACCTAACA | 55 | GTCCCCTCCCACTACAAATGGA | 56 |
| 16 | CCCAGCGTTTACATGAGC | 57 | GGGTGTGAATAAGCCATATTG | 58 |
| 17 | CCCCACTTGGTTATTTATGAC | 59 | CCCCACCACAACTATTAGG | 60 |
| 18 | GCATTTAGCCTACCGTGGC | 61 | AAGCTGCTATGTGAGTGCCG | 62 |
| 19 | GAGAAGAACAGAGGCATGGCACTAC | 63 | GTATGTATGATGCGTATGTGG | 64 |
| 20 | TGGCACTACACCCTACCTTCCAT | 65 | ATGCGTATGTGGAGGTCAGAACAC | 66 |
| 21 | CCCAGGCTATTCCCAGTGTG | 67 | CAGTCTCCCTGCCCTGTCTTA | 68 |
| 22 | CATCTGACACCATTTCCTGA | 69 | CATAGAGCATCTTCTGACGG | 70 |
| 23 | CTGTGAGTACTTCGTGCAG | 71 | AGAGTGCCAAGTCACTGTTAC | 72 |
| 24 | AAGTAAGTCGTGAGGCTTGT | 73 | AGGCATTTATCCATGAGGTAC | 74 |
| 25 | GTGCGCACGTTGAAGTATGTGA | 75 | ACCAACCGCCAGCTTTATTGAC | 76 |
| 26 | GGCAGCTACTGCAGGAACTG | 77 | CGCCTAAGCACAATTCCATT | 78 |
| 27 | ATGCCAACCTGAAGAACTAAC | 79 | CAGATCAGACGCTCTCCCACACT | 80 |
| 28 | TCAGAGAGATGCAGCCTTTGACTAA | 81 | ACAGCCTGTTTCCTCTCAGCATT | 82 |
| 29 | GTGGTGCACACACGTGTAGGC | 83 | TGCTGTTACCGACCAGGAAACA | 84 |
| 30 | CTGCCCATTCCTTAATTCAC | 85 | AGCCAGAACATCAGGGTTAG | 86 |
| 31 | TCACATGAGCCTTTGCAAGACA | 87 | GGCTGCGGATGACTGATCAG | 88 |
| 32 | CAGGCCACACTCACAATAGAAGATG | 89 | TGAGACTTCCCACGTGAGGATAAA | 90 |
| CD3e | GGCCCGGATTTCCTCAGTTA | 91 | GCCATAGTAGGATGAAGGAG | 92 |

Expression

| | | | | |
|---|---|---|---|---|
| Mina53 | CAGTAGGGCCAGATAAGAATCCAT | 93 | CATGTGCATCTGCCTCACATT | 94 |
| I14 | CTTATCGATGAATCCAGGCATCG | 95 | CATCGGCATTTTGAACGAACGAGGTCA | 96 |
| I12 | ATGTACAGCATGCAGCTCGCATC | 97 | GGCTTGTTGTTGAGATGATGCTTTGAC | 98 |
| Ifng | GGATGCATTCATGAGTATTGC | 99 | CCTTTTCCGCTTCCTGAGG | 100 |
| Hprt | GAGGGTAGGCCTATGGCT | 101 | GTTGGATACAGGCCAGACTTTGTTG | 102 |
| Bactin | ACTATTGGCAACGAGCGGTTC | 103 | GGATGCCACAGGATTCCATACC | 104 |

Evidence obtained in these ways supports the conclusion that Mina53 is a gene in the chromosome 16 Dice 1.2 genetic interval that is also an interleukin-4 (IL4 or IL-4) regulatory locus. These lines of evidence include:

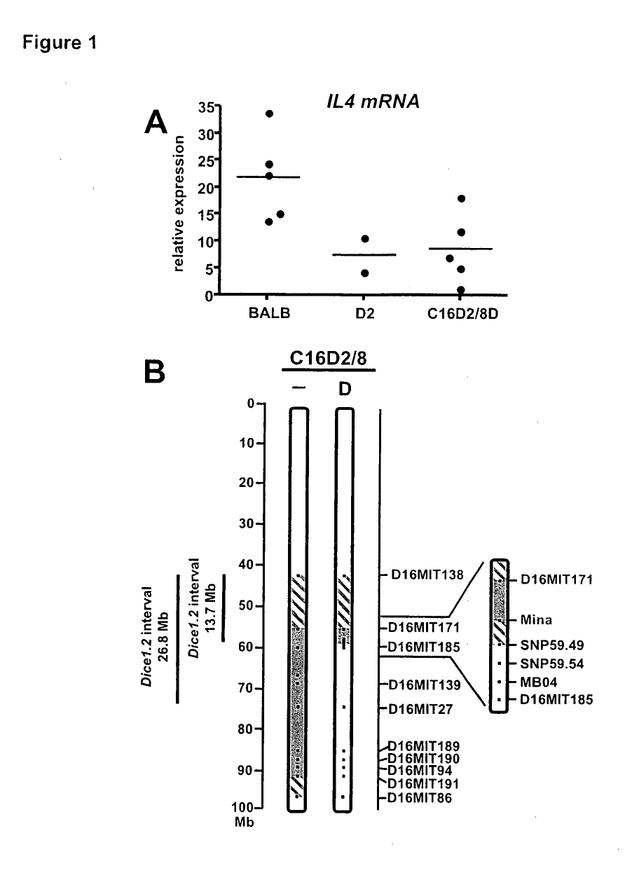

1. The effect of narrowing the Dice1.2 genetic interval. Previously Dice1.2 was identified as an IL4 regulatory locus controlling TH2 bias, the propensity of progenitor T cells to develop into IL4 secreting TH2 cells (Bix et al., J Exp Med, 188:2289-2299, 1998; and Baguet et al., J Exp Med, 200:1605-1612, 2004). Using interval specific congenic mice the chromosome 16 Dice1.2 genetic interval was narrowed to 26.8 Mb (Baguet et al., supra, 2004). To further narrow this interval, as described herein a number of new inbred congenic strains were generated that dissect the Dice1.2-spanning B10.D2-derived congenic interval contained in strain C16D2/8. Splenic CD4 T cells from one of these strains (C16D2/8D) were primed for 16 hours before analyzing cells for IL4 expression by quantitative RT-PCR. T cells from C16D2/8D produced IL4 at levels similar to control B10.D2 (FIG. 1A), indicating that Dice1.2 is contained in the C16D2/8D congenic interval. These results update and constrain Dice1.2 to a 13.9 Mb interval (FIG. 1B, D16MIT138 and SNP59.49) containing 110 predicted or known genes (retrieved from the website of the Mouse Genome Database, Mouse Genome Informatics, The Jackson Laboratory, Bar Harbor, Me.; and Eppig et al., Nucleic Acids Res, 35:D630-D637, 2007). Excluding 29 of these gene, which are olfactory receptor genes, leaves 81 Dice1.2 candidate genes.

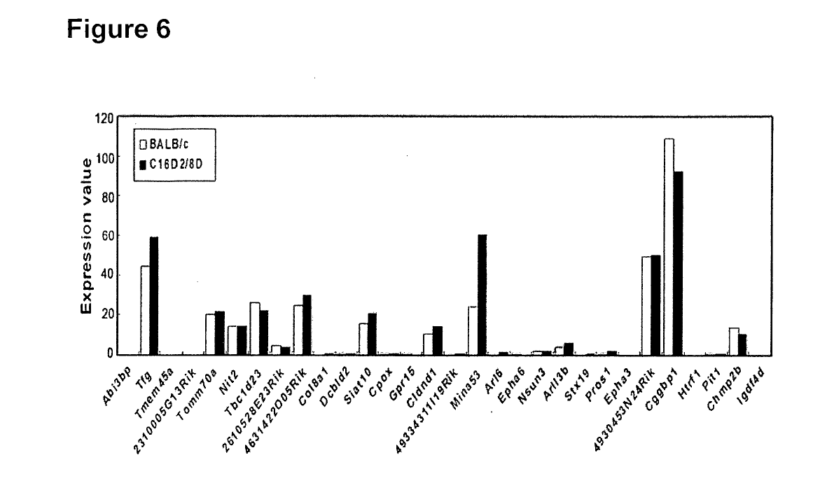

2. Expression profiling the Dice1.2 interval. The polymorphisms in Dice1.2 underlying genetic variability in TH2 bias may reside in coding and/or regulatory sequence. In order to test for the latter, genes in the Dice1.2 interval were interrogated by comparative expression profiling using mRNA isolated from BALB/c and C16D2/8D CD4 T cells stimulated by TCR cross-linking for 16 hours. Of the interrogated Dice1.2 candidate genes (30 of 81), half were undetectable and 14 showed similar expression levels in BALB/c and C16D2/8D. As shown in FIG. 6, the only gene displaying differential expression was Mina53, with levels in C16D2/8D two to three times higher than in BALB/c.

Mina53 mRNA expression in various tissues and cell types was determined by the RIKEN Research Center for Allergy and Immunology RefDIC (Reference Database of quantitative mRNA and protein data specialized for Immune and Immune-related Cells) database. Among immune cells, expression of Mina53 was highest in activated CD4 T cells, although it could be detected at moderate levels in LPS-stimulated bone marrow-derived dendritic cells and very low levels in B cells, macrophages, mast cells and CD8 T cells. Thus, Mina53 was identified during development of the present invention as a Dice1.2 candidate gene.

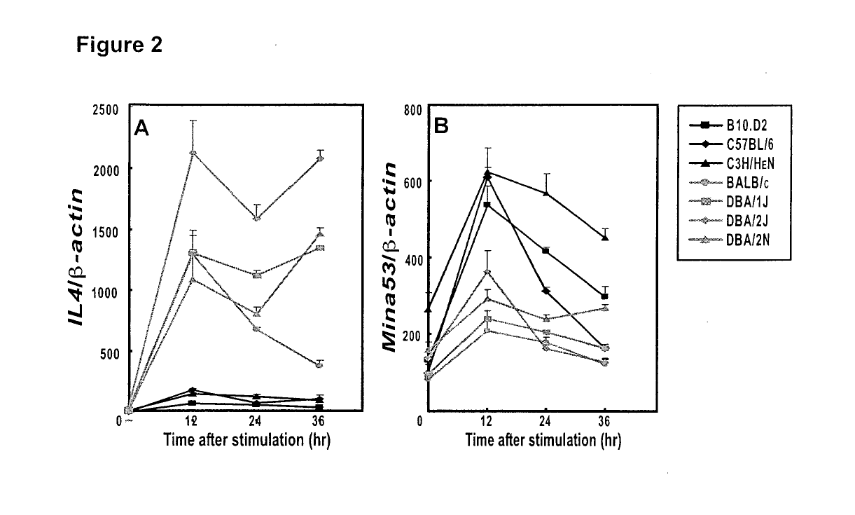

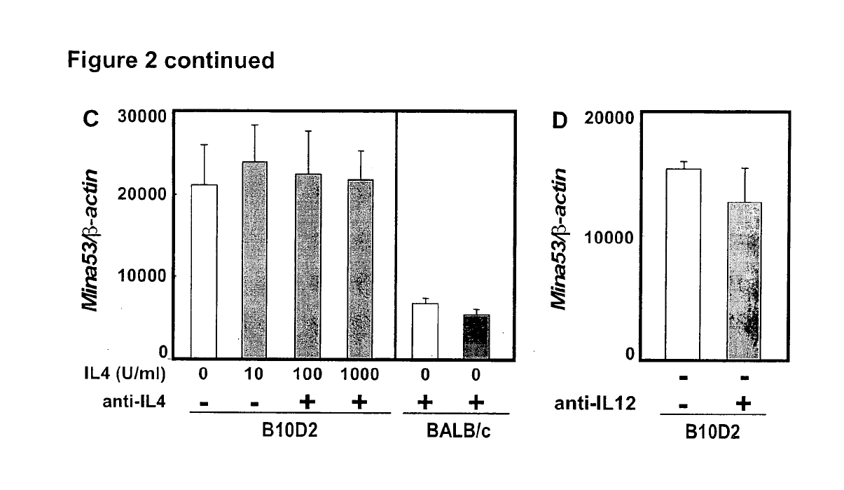

3. An inverse correlation between Mina53 and IL4 in strains with TH2 bias phenotypes. Quantitative RT-PCR was used to analyze the level and expression kinetics of IL4 and Mina53 in recently activated progenitor T cells isolated from a panel of independent inbred mouse strains representing high and low TH2 bias phenotypes. The kinetics of IL4 and Mina53 induction were similar across the tested strains, rapidly reaching peak levels by at least 12 hours and falling off over the ensuing 24 hours (FIGS. 2A and 2B), consistent with Mina53 being a regulator of IL4 expression. Although displaying similar kinetics of IL4 and Mina53 induction, the tested strains varied significantly in the magnitude of this induction. In particular, the magnitude of each response segregated the tested strains into two discrete groups with inversely correlated levels of IL4 and Mina53 expression. In B10.D2, C57BL/6 and C3H/HeN high Mina53 correlated with low IL4; while in BALB/c, DBA/1J, DBA/2J and DBA/2N the converse was true (FIGS. 2A and 2B). Thus as determined during development of the present invention, both kinetic and transcript level analyses are consistent with MINA acting in an inhibitory pathway to regulate the production of IL4 from progenitor T cells.

4. Mina53 level unaffected by IL4 and IL12 signaling pathways. To eliminate the possibility that IL4 inhibits Mina53 transcription, the expression of Mina53 mRNA in B10.D2 CD4 T cells in the presence and absence of exogenously added IL4 and IL12 was determined. Neither IL4 nor IL12 were capable of altering the expression of Mina53 (FIGS. 2C and 2D). Moreover, neutralization of IL4 with an IL4-specific antibody was incapable of enhancing Mina53 expression in BALB/c CD4 T cells. Together, these results demonstrate that Mina53 transcription is independent of IL4 and IL12 signaling pathways. Thus MINA53 has been determined to play an upstream causative role in modulating IL4 expression, by suppression of progenitor T helper IL4 expression thereby altering TH2 bias.

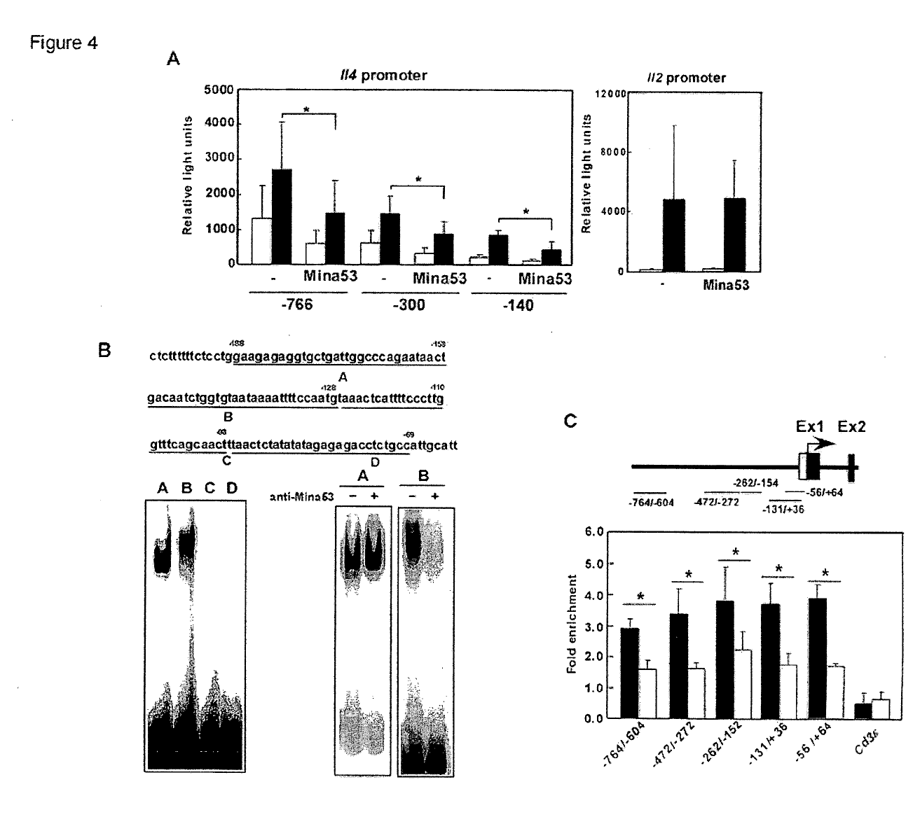

5. Mina53 binds to and represses the IL4 promoter. To test whether MINA53 directly inhibits IL4 transcription, a transient reporter assay using T cell hybridoma 68-41 was utilized. In this system, Mina53 over-expression inhibited TCR activation-dependent luciferase activity driven by the promoter for IL4 but not for IL2 (FIG. 4A). To localize the MINA53 responsive region, deletion mutants of the 766 bp IL4 promoter were tested. As shown in FIG. 4A, neither truncation of the IL4 promoter to 300 bp nor to 140 bp was sufficient to eliminate its sensitivity to MINA53-dependent repression. Thus, a target site for the repressive activity of MINA53 is likely to occur within the first 140 bp of the IL4 promoter (upstream of the transcriptional start site). To further refine the MINA53 responsive region, EMSA analysis was done. Nuclear extracts isolated from TCR-stimulated B10.D2 CD4 T cells were tested against a series of end-labeled oligonucleotide probes spanning the 140 bp IL4 promoter. Nucleoprotein complexes were formed with probes A and B, but not C or D (FIG. 4B). However, only the probe B complex was sensitive to inhibition by a MINA53 specific antibody. Thus as determined during development of the present invention, MINA53 binds to the IL4 promoter over the 30 bp probe B region extending from −157 to −128, 5′-GACA ATCTGGTGTA ATAAAATTTT CCAATG (SEQ ID NO:114).

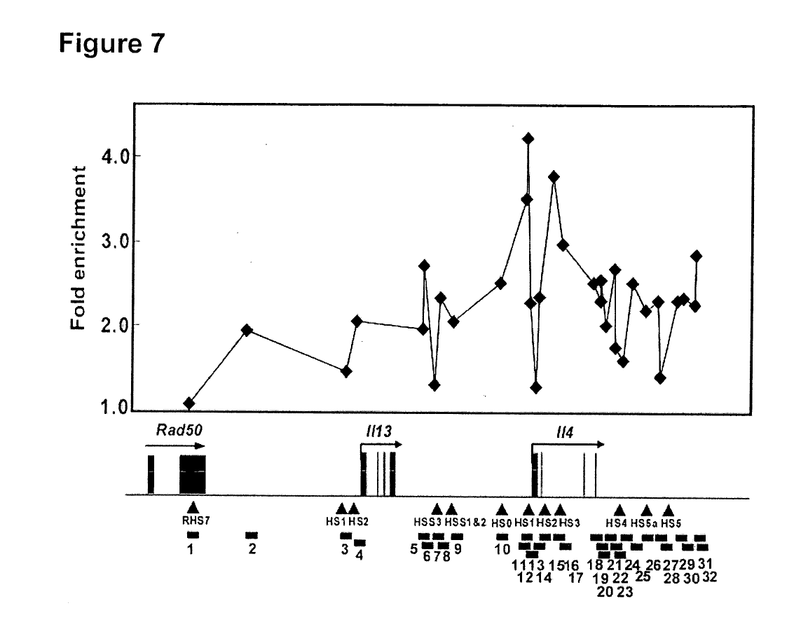

To determine whether MINA53 binds to the IL4 promoter in vivo, anti-MINA53 chromatin immunoprecipitation (ChIP) analysis of the entire TH2 locus was performed using 24 hour activated B10.D2 CD4 T cells. As shown in FIG. 7, a strong peak of MINA53 binding was detected at the IL4 promoter. Results of ChIP analysis focused on the IL4 promoter region were consistent with the peak binding activity detected by EMSA in the −157 to −128 region (FIG. 4C). Moreover, the magnitude of the binding activity was consistently higher in nuclei isolated from B10.D2 as compared to BALB/c, correlating with the relative expression level of Mina53 in these two strains. Together, these data support a model in which MINA binds to the proximal IL4 promoter and exerts an inhibitory influence on its transcription. Nonetheless understanding of the mechanisms involved is not necessary in order to make and use the present invention.

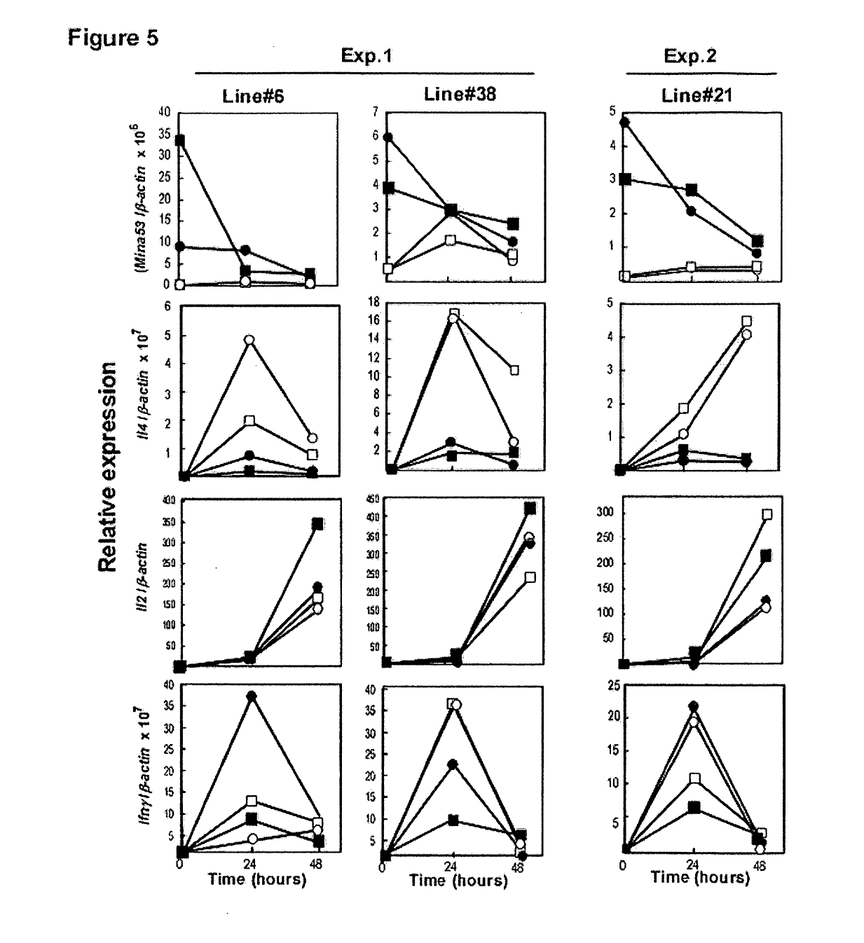

To test whether MINA53 could inhibit IL4 transcription in progenitor T helper cells, three independent BDF1XB6 mouse lines were generated expressing a Mina53 transgene driven by a T cell-specific proximal Lck promoter/IgM enhancer (Allen et al., Mol Cell Biol, 12:2758-2768, 1992). After three backcrosses to BALB/c (low MINA53, high IL4 phenotype), Mina53 transgenic lines displayed normal T cell development and activation marker expression. In vitro activated progenitor T helper cells from transgenic and littermate controls were compared for IL4 and Mina53 expression, as measured by quantitative real time PCR. Control littermates exhibited the expected rapid and transient induction of Mina53. By contrast, transgenic cells displayed extremely high basal levels of Mina53 that, upon activation, rapidly diminished to levels that were comparable to or higher than peak levels exhibited by control cells (FIG. 5).

To determine the effect of transgenic Mina53 over-expression on cytokine gene expression, the magnitude and kinetics of IL2, IL4 and IFNγ induction was compared between transgenic and littermate control progenitor T helper cells. Expression of IL2 and IFNγ were unaffected by transgenic Mina53 over-expression. By contrast, IL4 expression in Mina53 transgenic cells was dramatically impaired in comparison to littermate controls (FIG. 5). Thus MINA53 is a specific inhibitor of IL4 expression in progenitor T helper cells.

The implication that Mina53 genotypes in mice are associated with TH2 biases, and that MINA genotypes can be found in humans in association with IL4-mediated diseases was tested as follows:

1. Human subjects. All subjects with asthma were diagnosed according to the American Thoracic Society criteria as described (Am Rev Respir Dis, 85, 1962; and Harada et al., Am J Respir Cell Mol Biol, 36:491-496, 2007). 484 adults with asthma (mean age 50.9, 20-75 years; male:female ratio=1.0:1.32; atopic asthma 94.8%) were recruited, and their age, sex, serum total IgE level and clinical severity were recorded. The clinical severity of adult asthma was classified according to the criteria of the National Institutes of Health/Global Initiative for Asthma by physicians who were experts in allergic diseases, and was defined by symptoms at the time of entry into the study. The distribution of subjects was as follows: step 1, mild intermittent 2.3% (11 individuals); step 2, mild persistent 54.0% (262 individuals); step 3, moderate persistent 26.8% (130 individuals); and step 4, severe persistent 16.9% (82 individuals). The mean of serum total IgE (IU/ml) of adult asthma was 688.

A total of 717 healthy individuals who had neither respiratory symptoms nor a history of asthma-related diseases (mean age 49.3, 20-75 years; male:female ratio=2.59:1.0) were recruited by detailed physicians' interviews about whether they had been diagnosed with asthma, atopic dermatitis and/or nasal allergies. No lung function or serum IgE data are available for this population. All individuals were Japanese and gave written informed consent to participate in the study in accord with the rules of the process committee at the SNP Research Center, The Institute of Physical and Chemical Research (RIKEN).

2. Case/control statistical analysis. Pairwise linkage disequilibrium (LD) was calculated as Df/LOD and r₂ by using the Haploview 3.2 program available from the Broad Institute Haploview website (Barrett et al., Bioinformatics, 2005 Jan. 15 [PubMed ID: 15297300]). Five SNPs (rs3886138, rs9877544, rs832082, rs1532206 and rs2172257) were selected for association studies that captured all 9 alleles (r₂>0.92). Allele frequencies were calculated and agreement was tested with Hardy-Weinberg equilibrium using a chi² goodness of fit test at each locus. To test the association between MINA variants and bronchial asthma, differences in allele frequency and genotype distribution of each polymorphism were compared between case and control subjects using a contingency chi-square test with one degree of freedom (DF). Odds ratios (ORs) with 95 percent confidence intervals (95% CI) were also calculated. Associations between serum total IgE levels and clinical severity of subjects with adult asthma and five tested variants were investigated. During development of the present invention, high IgE levels were defined as those values in the 75^{th} percentile or higher for total IgE. The 75^{th} percentile value of total IgE in patients with adult asthma was 533 IU/ml. Statistical significance was defined at the standard 5% level.

3. Single nucleotide polymorphism (SNP) analysis of the mouse Mina53 and human MINA loci. BALB/c, B10.D2, C3H/HeN, DBA/2J were genotyped for SNPs in a 6.5 kb region extending 5′ from just before the initiation codon of Mina53 and compared with the published C57BL/6J sequence (GENBANK NO. AC154854). Likewise the MINA locus of human subjects was genotyped for SNPs and compared with the published MINA genomic sequence (GENBANK NT_{—}005612.15).

TABLE 2-1
Mina53 Primers for Mouse Genotyping

| | | SEQ | | SEQ |
|---|---|---|---|---|
| SNP | Forward primer 5′ to 3′ | ID | Reverse primer 5′ to 3′ | ID |
| SNP59.49 | ACTCATCACTAGGTGAGCGACACT | 21 | AGGCGTGTATGTGTCAGCCTTTCT | 22 |
| SNP59.54 | TAACAGCAAGCCACTCTTCGTCCT | 23 | TTGCCACCATGTCTTTGCTCTGTG | 24 |
| MB04 | AATATCTTTGGGCTTCCTACTGC | 25 | TTCAATTGCTTTCATTTGGATCT | 26 |

TABLE 2-2
MINA Primers and Probes for Human Genotyping

| | | SEQ | | SEQ |
|---|---|---|---|---|
| SNP | | ID | | ID |
| | Forward primer 5′ to 3′ | | Reverse primer 5′ to 3′ | |
| rs3886138 | CAGCATCATTGTGACTGGAGGTA | 01 | TCCAGCCTGGGCATCAG | 02 |
| rs9877544 | TGTCAGACCAAGTTATGATTTGAATTCAGA | 03 | CAAGGACTAGCTCAGCGAGAAG | 04 |
| rs1532206 | CCCTCCGGGTTGAGACTCA | 05 | CCTGGCGGCCTCACT | 06 |
| rs832082 | CCAGCCTGTTATTTCTTCTCTGTCT | 07 | TGGGAATTGCCTACACAAAATGGATAT | 08 |
| rs2172257 | TGTTGTTGATGGCCTAATGCA | 09 | CCCATCATGTGTGTCTCTCTACTATTC | 10 |
| | VIC probe 5′ to 3′ | | FAM probe 5′ to 3′ | |
| rs3886138 | AAAGTGCAACCCTATCCT | 12 | AAAGTGCAACCCTGTCCT | 12 |
| rs9877544 | CATTTTATTGACAGTGTCCAT | 14 | TTTTATTGACGGTGTCCAT | 14 |
| rs1532206 | CTTTGTGACTTAATATTATATC | 16 | TTTGTGACTTAATGTTATATC | 16 |
| rs832082 | TGATAACACAGGAAGAAG | 18 | AACACGGGAAGAAG | 18 |
| rs2172257 | AAAGGATGAAGCTCAAGAA | 20 | CAAAGGATGAAACTCAAGAA | 20 |

Several lines of evidence emerging from the foregoing confirm the implication that Mina53 genotypes in mice are associated with TH2 biases, and that MINA genotypes can be found in humans in association with IL4-mediated diseases was tested as follows:

1. Mina53 polymorphisms predict TH2 bias. To examine the molecular basis for differential Mina53 expression between high (C57BL/6, C3H/HeJ) and low (DBA/1J, DBA/2J and DBA/2N) TH2 biased strains, the 5′ end of the Mina53 genomic locus was sequenced (including intron 1 and 1304 bp of the promoter) from C57BL/6, B10.D2/OsnJ, C3H/HeN, BALB/c and DBA/2J mice. Twenty-one noncoding SNPs were identified that defined two distinct haplotypes correlating with the expression level phenotypes of MINA53 and IL4 (FIG. 3). Thus, Mina53 haplotypes are a useful genetic marker for TH2 bias and are contemplated to contain the regulatory SNP(s) controlling Mina53 expression level phenotypes.

2. MINA SNPs are associated with both bronchial asthma and allergic rhinitis. Given that Mina53 regulates IL4 expression in progenitor mouse T helper cells, the inventors examined whether MINA (the orthologous human gene, 76% homology) plays a similar role in humans. Human populations stratified by TH2 bias or IL4 expression phenotypes are not available for study. For this reason, bronchial asthma (BA) and allergic rhinitis (AR), two globally important TH2-dependent diseases, were studied. Case control studies of several well-characterized Japanese populations were performed. For BA the following groups were tested: 484 adults with BA, 435 adults with atopic BA; and 714 controls. For AR the following groups were tested: 296 individuals with RAST+ (Derf or Derp) AR, and 306 controls. In the HapMap Japanese data set there were nine MINA SNPs with minor allele frequencies>10% (Table 2-3). Using the Tagger tool from the Haploview 3.2 software package, five SNPs (rs3886138, rs9877544, rs832082, rs1532206 and rs2172257; r2>0.91) were identified that captured all 9 alleles. Bonferroni correction was done for the number of variants investigated and the data were analyzed for associations with BA and related phenotypes, as well as with AR.

TABLE 2-3
Locations and Allele Frequencies of Human MINA Polymorphisms

| | | Orien- | | | |
|---|---|---|---|---|---|
| | | tation/ | Minor | | SEQ |
| SNP name^{a} | Location | Strand | Allele (ν)^{c} | Sequence | ID NO |
| rs3886138^{b} | 5′ | fwd/ | A (0.28) | GCATCAGGCTGCAAAAGTGCAACCCT | 105 |
| | | top | | [A/G] | |
| | | | | TCCTACTCCTACCTCCAGTCACAAT | |
| rs4857304 | Intron 1 | | G (0.16) | AGACTGATCTAAAACATTTCTTTTTG | 106 |
| | | | | [G/T] | |
| | | | | CTCAGAGATATGTTAATCAATCCAG | |
| rs9877544^{b} | Intron 2 | | G (0.16) | AGCTCAGCGAGAAGCATTTTATTGAC | 107 |
| | | | | [A/G] | |
| | | | | GTGTCCATATTTTCTGAATTCAAAT | |
| rs832082^{b} | Intron 2 | | A (0.10) | AGCAATGGGAACAGAAATGATAACAC | 108 |
| | | | | [A/G] | |
| | | | | GGAAGAAGGTACAGACAGAGAAGAA | |
| rs9879532 | Intron 5 | | C (0.19) | ATGCTCCAAGAGTCTCCTCTGCAAAC | 109 |
| | | | | [C/T] | |
| | | | | TACAATAGAATTTATCACACCATCT | |
| rs1532206^{b} | Intron 5 | | G (0.39) | GCGGCCTCACTGCTTTGTGACTTAAT | 110 |
| | | | | [A/G] | |
| | | | | TTATATCCAACCCTGCATCTTTAAT | |
| rs2172257^{b} | Exon 9, | | C (0.18) | ATAGATGTACACCATCTTTTCTTGAG | 111 |
| | T385A | | | [C/T] | |
| | | | | TTCATCCTTTGGGGAAAAAATAAAT | |
| rs4857302 | 3′ | | A (0.17) | TGTGCATTTCTCCCTATAGTGGAAAG | 112 |
| | | | | [A/C] | |
| | | | | ATACTCAGATCTGGTACTACTGCCG | |
| rs2201151 | 3′ | | G (0.15) | ATTAATCTATGCAGATTTACTAAAGG | 113 |
| | | | | [G/T] | |
| | | | | AATAAGAGTGAAACTGATATAACTA | |

^{a}Number from the National Center for Biological Information dbSNP.
^{b}SNPs genotyped during development of the present invention.
^{c}Identity and (frequency) of the minor allele.

TABLE 2.4
Association of MINA53 SNPs with Asthma

| | | | | Asthma | Control | | Asthma | Control |
|---|---|---|---|---|---|---|---|---|
| | | | | n = 484 | n = 717 | | n = 484 | n = 717 |
| SNP name | SNP^{a} | location | genotype | (%) | (%) | allele | (%) | (%) |
| rs3886138 | −3108 | 5′ | CC | 275 (56.9) | 403 (56.4) | C | 727(75.3) | 1079(75.5) |
| | C/T | | CT | 177 (36.6) | 273 (38.2) | T | 239(24.7) | 351(24.5) |
| | | | TT | 31 (6.4) | 39 (5.6) | | | |
| rs9877544 | 6228 | Int2 | TT | 290 (59.9) | 416 (58.0) | T | 749(77.4) | 1096(76.4) |
| | T/C | | TC | 169 (34.9) | 264 (36.8) | C | 219(22.6) | 338(23.6) |
| | | | CC | 25 (5.2) | 37 (5.2) | | | |
| rs832082 | 9643 | Int2 | CC | 349 (73.0) | 527 (74.0) | C | 814(85.1) | 1232(86.5) |
| | C/T | | CT | 116 (24.3) | 178 (25.0) | T | 142(14.9) | 192(13.5) |
| | | | TT | 13 (2.7) | 7 (1.0) | | | |
| rs1532206 | 20305 | Int5 | TT | 147 (30.4) | 208 (29.4) | T | 514(53.2) | 785(55.5) |
| | T/C | | TC | 220 (45.5) | 369 (52.2) | C | 452(46.8) | 629(44.5) |
| | | | CC | 116 (24.0) | 130 (18.4) | | | |
| rs2172257 | 26257 | Ex9 | AA | 251 (52.1) | 365 (51.1) | A | 690(71.6) | 1017(71.2) |
| | A/G | T385A | AG | 188 (39.0) | 287 (40.2) | G | 274(28.4) | 411(28.8) |
| | | | GG | 43 (8.9) | 62 (8.7) | | | |

| | | | P value, OR (95% C.I.) | | | |
|---|---|---|---|---|---|---|
| SNP name | SNP^{a} | location | Allelic | Genotypic | Dominant | Recessive |
| rs3886138 | −3108 | 5′ | 0.913 | 0.720 | 0.845 | 0.485 |
| | C/T | | | | | |
| rs9877544 | 6228 | Int2 | 0.590 | 0.792 | 0.512 | 0.997 |
| | T/C | | | | | |
| rs832082 | 9643 | Int2 | 0.345 | 0.073 | 0.700 | 0.022, 2.82 |
| | C/T | | | | | (1.12-7.11) |
| rs1532206 | 20305 | Int5 | 0.267 | 0.029 | 0.707 | 0.019, 1.40 |
| | T/C | | | | | (1.06-1.86) |
| rs2172257 | 26257 | Ex9 | 0.849 | 0.917 | 0.746 | 0.887 |
| | A/G | T385A | | | | |

^{a}Numbering according to the genomic sequence of MINA53 (NT_005612.15). Position 1 is the A of the initiation codon.
Significant associations are bold and red.

As shown in Table 2-4, a significant association was detected between asthma and the rs832082 and rs1532206 genotypes (P=0.022 and P=0.019, respectively). In each case there was an excess of minor allele homozygotes, consistent with a recessive model of inheritance. Thus, two distinct SNPs in the MINA locus (rs832082 in intron 2; and rs1532206 in intron 5) are each associated with susceptibility to asthma.

As shown in Table 2-5, a significant association was detected between the rs3886138 genotype and RAST+ (Derf or Derp) AR (P<0.0497). Again, in each case there was an excess of minor allele homozygotes. Thus, SNP rs3886138 (located 3108 bp upstream of the MINA transcriptional start site) is associated with susceptibility to AR.

In the same populations, MINA polymorphisms were tested for an association with asthma severity and serum IgE (a classic marker of atopic diseases including asthma). As shown in Table 2-6, a significant association was also detected between asthma severity and the minor allele of rs1532206 (P=0.021) and the rs2172257 genotype (P=0.019). Finally, as shown in Table 2-6, in adult bronchial asthmatics a significant association was detected between serum total IgE and both the rs3886138 genotype (P=0.013) and its minor allele (P=0.024). Together these results demonstrate that genetic variation in the human MINA locus is significantly associated with a biomarker of atopic disease (serum total IgE) and with the incidence and severity of asthma, a globally important TH2-dependent disease.

TABLE 2-5
Association of MINA SNPs with Allergic Rhinitis (RA)*

| | | | | | | *Major | *Minor |
|---|---|---|---|---|---|---|---|
| Allergic | | | | Genotype | Allele | vs. | vs. |
| Rhinitis | SNP | location | SNP name | P value | P value | others P | others P |
| normal vs. | −3108 A/G | 5′g | rs3886138 | 0.106 | 0.609 | 0.802 | 0.0497 |
| Derf or | 6228 A/G | int2 | rs9877544 | 0.980 | 0.855 | 0.899 | 0.850 |
| Derp | 9643 A/G | int2 | rs832082 | 0.644 | 0.468 | 0.619 | 0.368 |
| RAST+ | 20305 A/G | int5 | rs1532206 | 0.976 | 0.872 | 0.957 | 0.826 |
| | 26257 C/T | ex9, T385A | rs2172257 | 0.488 | 0.516 | 0.838 | 0.234 |

*Major = major allele homozygotes; and Minor = minor allele homozygotes.

TABLE 2-6
Association Of MINA SNPs with Asthma Severity (S TABLE 3)

| | | | Child | | Adult |
|---|---|---|---|---|---|
| | | | Atopic | Adult BA | Atopic |
| SNP | location | SNP name | P value | P value | P value |
| −3108 A/G | 5′g | rs3886138 | 0.1740 | 0.3137 | 0.3144 |
| 6228 A/G | int2 | rs9877544 | 0.2865 | 0.7820 | 0.8191 |
| 9643 A/G | int2 | rs832082 | 0.8948 | 0.4548 | 0.5082 |
| 20305 A/G | int5 | rs1532206 | 0.5120 | 0.0155 | 0.0414 |
| 26257 C/T | ex9, T385A | rs2172257 | 0.5370 | 0.2681 | 0.3010 |

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the relevant fields are intended to be within the scope of the claims.

### EXPERIMENTAL

The following are examples of are provided in order to demonstrate and further illustrate methods used in developing the present invention, which may be of use in certain preferred embodiments and aspects of the present invention and are not to be construed as limiting the scope thereof.

### Example 1

### Myc-Induced Nuclear Antigen of 53 Kilodaltons (Mina53) Controls TH2 Bias

This example describes the identification of Mina53 as a gene in the chromosome 16 Dice 1.2 genetic interval as an interleukin-4 (IL4 or IL-4) regulatory locus.

Mice. BALB/c, C57BL/6, C3H/HeN, B10.D2, DBA/2J, DBA/1J and DBA/2N mice were purchased from Jackson Lab (Bar Harbor, Me.), Taconic Farms (Hudson, N.Y.), CLEA Japan, Inc. (Tokyo, Japan), and Charles River Laboratory (Wilmington, Mass.). Mice were bred and maintained in specific pathogen-free conditions and in accordance with the guidelines of the Institutional Animal Care and Use Committees of the RIKEN Yokohama Institute (Yokohama, Japan) and St. Jude Children's Research Hospital (Memphis, Tenn.).

Establishment of congenic mouse strains and phenotypic screening for TH2 cell bias. Generation of congenic strain C16D2/8 was described previously (Baguet et al., J Exp Med, 200:1605-1612, 2004). First BALB.D2c16 mice were generated from a founding (BALB/c×B10.D2) F1 backcrossed to BALB/c and containing a B10.D2 derived congenic interval spanning the majority of chromosome 16. Nine congenic lines (C16D2/1-9) were generated by backcrossing BALB.D2c16 to BALB/c. After screening these congenic lines for the TH2 cell bias trait, C16D2/8 was selected for further analysis. Backcrossing C16D2/8 to BALB/c generated c16D2/8-a and C16D2/8-b congenic lines. Splenic CD4 T cells, isolated by MACS CD4 T cell Isolation kit from Miltenyi Biotec (Auburn, Calif.), were stimulated with plate-bound anti-TCR (1 μg/ml) and anti-CD28 (37N.51.1, 10 μg/ml) antibodies for 16 hours prior to harvesting RNA with TRI REAGENT from Sigma (St. Louis, Mo.) and determining 114 and Hprt expression by reverse transcriptase real time PCR as described (Baguet et al., Proc Natl Acad Sci USA, 101:11410-11415, 2004). In subsequent experiments, splenic CD4 T cells were isolated by iMag CD4 T cell Isolation kit from Sigma, and stimulated with plate-bound anti-TCR (1 μg/ml) and soluble anti-CD28 (PV-1, 10 μg/ml) antibodies for 24 hours prior to harvesting RNA with TRI REAGENT from Sigma, and expression analysis by reverse transcriptase real time PCR as described (Seki et al., J Immunol, 172:6158-6166, 2004).

Generation of Mina53 Transgenic Mice. To establish Mina53 transgenic mice, a B10.D2 Mina53 cDNA was cloned downstream of the Lck proximal promoter and Eμ enhancer in the p1026X expression vector (Attar et al., Mol Cell Biol, 18:477-487, 1998). PCR of genomic tail DNA identified ten independent BDF1XB6 transgenic founders. Transgene copy number was determined by Southern blot analysis. Three founders harboring over 10 copies of the transgene were selected and backcrossed for three generations to BALB/c prior to further analysis. Protein expression in splenic CD4 T cells was confirmed by immunoblot analysis with anti-Mina53 antibody.

Reagents and antibodies. Anti-TCRβ antibody was purified from hybridoma H57.597. Anti-CD28 was purified, respectively, from hybridomas PV-1 and 37N51.1, kind gifts respectively from Ryo Abe (Science University of Tokyo) and James Allison (Memorial Sloan Kettering Cancer). Anti-IL4 was used as 10% culture supernatant from hybridoma 11B11, a kind gift from William Paul (NIH). Anti-IL12 was used as 10% culture supernatant from hybridoma C17.8, a kind gift from Georgio Trinchieri (NCI). Anti-Mina53 antibody (clone M532 described by Teye et al., Am J Pathol, 164:205-216, 2004; and Tsuneoka et al., Clin Cancer Res, 10:7347-7356, 2004) was purchased from Zymed (South San Francisco, Calif.).

Preparation of RNA and expression profiling. CD4 T cells were isolated from BALB/c or congenic strains, and cells were stimulated with plate bound anti-TCR mAb (H57-597, 1 μg/ml) and soluble anti-CD28 mAb (37N.51.1, 10 μg/ml). After 24 hrs, total RNA was extracted with TRI REAGENT from Sigma and passed through RNeasy mini-columns from QIAGEN (Valencia, Calif.). cRNA were synthesized from the isolated RNA according to Affymetrix protocols. The biotinylated cRNAs were hybridized to Affymetrix Inc. (Santa Clara, Calif.) GeneChips (mouse Genome 430 version 2). The microarray data were globally normalized using the GeneSpring software package from Agilent Technologies (Santa Clara, Calif.).

Quantitative RT-PCR. Total RNA was extracted from cells and cDNA was synthesized using SuperScript III Reverse Transcriptase from Invitrogen Corp. (Carlsbad, Calif.). Real-time quantitative reverse transcription PCR analysis was performed on MX4000 from Stratagene (La Jolla, Calif.) and 7500 real-time PCR systems from Applied Biosystems (Foster City, Calif.). The PCR primers used are shown in Table 1-1. mRNA expression was normalized internally to either β-actin or Hprt.

Transient reporter assay. Mouse (strain B10.D2) Mina53 cDNA was inserted into pCMV10 from Sigma-Aldrich to generate the pCMV FLAG-Mina53 expression construct. T cell hybridoma, 68-41 cells were transfected with the IL4 or IL2 promoter luciferase reporter plasmid (pIL4 Lu or pIL2 Lu) in the presence of pCMV or pCMV FLAG-Mina53, and a pancreatic alkaline phosphatase (PAP) expression plasmid (pSVPAP) to control for transfection efficiency. Forty hrs after transfection, cells were stimulated for 12 hrs with plate-bound anti-TCR mAb and cell lysates were prepared by extraction with lysis buffer from Promega Corp. (Madison, Wis.). Luciferase activity measured with a Microplate luminometer (Promega) was normalized to PAP activity.

Electrophoretic mobility shift assay (EMSA). Nuclear proteins were extracted with CelLytic Nuclear Extraction Kit from Sigma. The nuclear proteins (10 μg) were incubated with 1×10⁴ cpm of end-labeled oligonucleotide probe for 30 min at room temperature. The samples were loaded on 4% polyacrylamide gel and run in low ionic strength buffer (1×TAE). The supershift assay was carried out by incubation with rabbit anti-Mina43 antibody.

Chromatin immunoprecipitation (ChIP) assay. The ChIP assay was performed as previously described (Koyanagi et al., J Biol Chem, 280:31470-31477, 2005). Briefly, DNA recovered from an aliquot of sheared chromatin was used as the input sample. The remaining chromatin was pre-cleared with protein A and protein G agarose from Amersham Biosciences (Piscataway, N.J.) and then incubated with anti-Mina Ab overnight at 4° C. Input DNA and DNA recovered after immunoprecipitation (IP) were quantified with a PicoGreen fluorescence assay kit from Molecular Probes (Eugene, Oreg.). Equivalent masses of IP and input DNA were compared by real time PCR. Data are presented as the ratio of IP to input CT values.

### Example 2

### Association of Mina53 Genotype with Bronchial Asthma and Allergic Rhinitis

This example describes exemplary methods employed in the identification of Mina53 genotypes in mice and their association with TH2 biases, and the identification of Mina genotypes in humans and their associated with IL4-mediated diseases.

Human subjects. All subjects with asthma were diagnosed according to the American Thoracic Society criteria as described (Am Rev Respir Dis, 85, 1962; and Harada et al., Am J Respir Cell Mol Biol, 36:491-496, 2007). 484 adults with asthma (mean age 50.9, 20-75 years; male:female ratio=1.0:1.32; atopic asthma 94.8%) were recruited, and their age, sex, serum total IgE level and clinical severity were recorded. The clinical severity of adult asthma was classified according to the criteria of the National Institutes of Health/Global Initiative for Asthma by physicians who were experts in allergic diseases, and was defined by symptoms at the time of entry into the study. The distribution of subjects was as follows: step 1, mild intermittent 2.3% (11 individuals); step 2, mild persistent 54.0% (262 individuals); step 3, moderate persistent 26.8% (130 individuals); and step 4, severe persistent 16.9% (82 individuals). The mean of serum total IgE (IU/ml) of adult asthma was 688.

A total of 717 healthy individuals who had neither respiratory symptoms nor a history of asthma-related diseases (mean age 49.3, 20-75 years; male:female ratio=2.59:1.0) were recruited by detailed physicians' interviews about whether they had been diagnosed with asthma, atopic dermatitis and/or nasal allergies. No lung function or serum IgE data are available for this population. All individuals were Japanese and gave written informed consent to participate in the study in accord with the rules of the process committee at the SNP Research Center, The Institute of Physical and Chemical Research (RIKEN).

Case/control statistical analysis. Pairwise linkage disequilibrium (LD) was calculated as Df/LOD and r₂ by using the Haploview 3.2 program available from the Broad Institute Haploview website (Barrett et al., Bioinformatics, 2005 Jan. 15 [PubMed ID: 15297300]). Five SNPs (rs3886138, rs9877544, rs832082, rs1532206 and rs2172257) were selected for association studies that captured all 9 alleles (r₂>0.92). Allele frequencies were calculated and agreement was tested with Hardy-Weinberg equilibrium using a chi² goodness of fit test at each locus. To test the association between MINA variants and bronchial asthma, differences in allele frequency and genotype distribution of each polymorphism were compared between case and control subjects using a contingency chi-square test with one degree of freedom (DF). Odds ratios (ORs) with 95 percent confidence intervals (95% CI) were also calculated. Associations between serum total IgE levels and clinical severity of subjects with adult asthma and five tested variants were investigated. During development of the present invention, high IgE levels were defined as those values in the 75th percentile or higher for total IgE. The 75th percentile value of total IgE in patients with adult asthma was 533 IU/ml. Statistical significance was defined at the standard 5% level.

Single nucleotide polymorphism (SNP) analysis of the mouse Mina53 and human Mina loci. BALB/c, B10.D2, C3H/HeN, DBA/2J were genotyped for SNPs in a 6.5 kb region extending 5′ from just before the initiation codon of Mina53 and compared with the published C57BL/6J sequence (GENBANK NO. AC 154854). Likewise the MINA locus of human subjects was genotyped for SNPs and compared with the published MINA genomic sequence (GENBANK NT_{—}005612.15).

We claim:

1. A method of determining an individual's risk of developing an atopic disease comprising detecting, in a nucleic acid sample from said individual, the presence of at least one genetic variation, at least one of which is associated in linkage disequilibrium with at least a first MINA-associated single nucleotide polymorphism (“SNP”), wherein detection of the presence of the at least one linkage disequilibrium-associated genetic variation indicates that said individual has or is predisposed to the development of an atopic disease.

2. The method of claim 1 wherein said SNP is a minor allele selected from the group consisting of rs3886138, rs1532206 and rs2172257, wherein the presence of the minor allele indicates an increased risk for atopic disease relative to the presence of a major allele.

3. The method of claim 2, comprising detecting said first MINA-associated SNP and further comprising detecting at least one other MINA-associated SNP wherein said first SNP and said at least one other SNP are in a haplotype, said haplotype comprising rs3886138G, rs1532206G and rs2172257T.

4. The method claim 1, wherein the at least one genetic variation consists of at least one copy of a chromosome 16 comprising SNPs rs3886138G, rs1532206G and rs2172257T or SNPs in linkage disequilibrium therewith.

5. The method claim 1 wherein the atopic disease is selected from the group consisting of bronchial asthma and allergic rhinitis.

6. The method of claim 1, wherein the nucleic acid sample comprises DNA.

7. The method of claim 1, wherein the nucleic acid sample comprises RNA.

8. The method of claim 1, wherein said detecting step is selected from the group consisting of size analysis; sequencing; hybridization; 5′ nuclease digestion; single-stranded conformation polymorphism; allele specific hybridization; primer specific extension; and oligonucleotide ligation assay.

9. The method of claim 8, wherein said size analysis is preceded by a restriction enzyme digestion.

10. The method of claim 1, wherein the nucleic acid sample is amplified.

11. The method of claim 10, wherein the nucleic acid sample is amplified by a polymerase chain reaction.

12. The method of claim 1, wherein the at least one polymorphism is detected by amplification.

13. The method of claim 12, wherein the at least one polymorphism is detected by a polymerase chain reaction.

14. The method of claim 1, wherein the at least one polymorphism is detected by sequencing.

15. The method of claim 1, wherein the at least one polymorphism is detected by amplification of a target region containing the at least one polymorphism; and hybridization with at least one sequence-specific oligonucleotides that hybridizes under stringent conditions to the at least one polymorphism and detecting the hybridization.

16. A method for selecting an appropriate therapeutic for an individual that has or is predisposed to developing an atopic disease, comprising the steps of: detecting whether the subject contains a MINA-associated SNP; and selecting a therapeutic that compensates for the MINA-associated genetic variation.

17. The method of claim 16 wherein said detecting is performed using a technique selected from the group consisting of allele specific oligonucleotide hybridization; size analysis; sequencing; hybridization; 5′ nuclease digestion; single-stranded conformation polymorphism; primer specific extension; and oligonucleotide ligation assay.

18. The method of claim 16 wherein said therapeutic is a modulator of MINA activity.

19. The method of claim 18, wherein the modulator of MINA activity is a protein, peptide, peptidomimetic, small molecule, nucleic acid or a nutraceutical.

20. The method of claim 16, wherein said detecting is performed using a technique selected from the group consisting of allele specific oligonucleotide hybridization; size analysis; sequencing; hybridization; 5′ nuclease digestion; single-stranded conformation polymorphism; primer specific extension; and oligonucleotide ligation assay.

21. The method of claim 16, wherein prior to or in conjunction with said detecting, the nucleic acid sample is subjected to an amplification step.

22. A method of screening for predisposition to an atopic disease in a subject comprising detecting, in a nucleic acid sample from said individual, the presence of at least one genetic variation, at least one of which is associated in linkage disequilibrium with at least a first MINA-associated single nucleotide polymorphism (“SNP”), wherein said SNP is a minor allele selected from the group consisting of rs3886138, rs1532206 and rs2172257, wherein the presence of the minor allele indicates an increased risk for atopic disease relative to the presence of a major allele.

23. The method of claim 22, wherein the sample is selected from the group consisting of blood, saliva, amniotic fluid, and tissue.

24. The method of claim 22, wherein said nucleic acid is selected from the group consisting of mRNA, genomic DNA, and cDNA.

25. A kit for determining the existence of or a susceptibility to developing an atopic disease in a subject, said kit comprising a reagent for specifically detecting a specific MINA allele containing a SNP variation in linkage disequilibrium with at least a first MINA-associated single nucleotide polymorphism (“SNP”).

26. The kit of claim 25, wherein said reagent comprises a first primer and a second primer that hybridize either 3′ or 5′ to the MINA gene, so that a polymorphism can be amplified.

27. The kit of claim 25, wherein the polymorphism is selected from the group consisting of rs3886138, rs1532206 and rs2172257.

28. The kit of claim 26, wherein said first primer and said second primer hybridize to a region in the range of between about 50 and about 1000 base pairs.

29. The kit of claim 25, which additionally comprises a detection means.

30. The kit of claim 25, which additionally comprises an amplification means.

31. The kit of claim 25, which further comprises a control.
